(19) United States
(12) Patent Application Publication
(10) Pub. No.: US 2011/0076665 A1
(43) Pub. Date: Mar. 31, 2011

(54) ELECTROMAGNETIC CONTROLLED BIOFABRICATION FOR MANUFACTURING OF MIMETIC BIOCOMPATIBLE MATERIALS

(76) Inventors: Paul Gatenholm, Blacksburg, VA (US); Rafael V. Davalos, Blacksburg, VA (US); Michael B. Sano, Blacksburg, VA (US)

(21) Appl. No.: 12/996,412

(22) PCT Filed: Jun. 5, 2009
(86) PCT No.: PCT/US2009/046407

§ 371 Date: Dec. 6, 2010

Related U.S. Application Data
(60) Provisional application No. 61/131,017, filed on Jun. 5, 2008.

(51) Int. Cl. A01N 1/00; C40B 50/06; C12M 1/00; C08B 1/00; C12N 5/00; B32B 23/00

(52) U.S. Cl. 435/1.1; 435/101; 435/283.1; 536/56; 435/395; 428/536

(57) ABSTRACT

The precise application of an electromagnetic field controls cell motion to guide extrusion and deposition of biopolymers produced by the cells. This controlled biofabrication process is used to fabricate two- and three-dimensional networks of biocompatible nanofibrils (such as cellulose) for use as biomaterials, tissue scaffolds to be used in regenerative medicine, coatings for biomedical devices, and other health care products.

### BACKGROUND OF THE INVENTION

1. Field of the Invention

The present invention generally relates to biocompatible materials, tissue engineering and regenerative medicine, implants, biomedical devices and health care products and, more particularly, to systems and methods for production and control of architecture and morphology of biomaterials which mimic tissues and organs using electromagnetic biofabrication.

2. Background Description

Regenerative medicine holds great promise for providing replacement tissue and organs, but there is an emerging need for new biomaterials with controlled architecture. The concept of engineering tissue using selective cell transplantation has been applied experimentally and clinically for a variety of disorders, including the successful use of engineered skin for burn patient and engineered cartilage for knee replacement procedures. However, the ability to generate mimetics with complex structures remains dependent upon the underlying scaffold that supports the cells and allows functional units of multiple cell types to interact and organize appropriately. The choice of biomaterial-scaffold is crucial to enable the cells to behave in the required manner to produce tissues and organs of the desired shape, size and mechanical properties.

Traditional manufacturing methods for biomaterials have limitations with regard to control of shape and size. Since top-down manufacturing methods are inadequate for manufacturing larger devices that can generate complex nano-sized features, there is an emerging interest in using biological systems for manufacturing. Biofabrication, the combination of biology and microfabrication, may be the future solution for the production of complex 3D architectures with nanoscale precision.

It has been previously demonstrated that bacteria can be magnetically manipulated to create complex magnetite nanoparticle chains or be ultrasonically processed to create hollow metal chalcogenide nanostructures, and genetically engineered viruses can be used to fabricate ordered arrays of quantum dots. Magnetic fields have also been used to disrupt assembly of nanofibers to produce amorphous material and magnetic alliteration of cellulose during biosynthesis (Brown M U.S. Pat. No. 4,891,317). A vast number of other potentially useful biological processes exist, and biological assembly can be affected by various stimuli such as electrical fields, magnetic fields, temperature, pH, or chemical gradients.

There remains an unrealized potential to overcome the limitations of material production for regenerative medicine and health care applications. For example, bio-fabrication of natural polymers like spider silk has been explored due to the outstanding strength of these polymers, including expression in mammalian milk and others (Wang, X., et al 2006, Fibrous proteins and tissue engineering, Materials Today 9, 44-53). The attempts to control the spinning process by manipulating spiders have unfortunately failed.

Cellulose, a natural polymer produced by most plants, is also produced in certain bacterial species to provide a protective environment for colony expansion. Typically, bacterial cellulose (BC) fibers are randomly deposited and assemble into nanofibrils that form a buoyant mat-like structure. BC has interesting properties in its wet, unmodified state but is also a versatile material that can be easily manufactured in various sizes and shapes. BC is an emerging biomaterial and several commercial products have already been registered (Biofill®, Gengiflex®). The use of microbial-derived cellulose in the medical industry has already been applied for liquid-loaded pads, wound dressings (Fontana, et al. 1990, Appl. Biochem. Biotechnol. 24, 253-264) and other external applications.

The advantage of BC is that it has unique biocompatibility, mechanical integrity, hydroexpansivity, and is stable under a wide range of conditions. The high water content of bacterial cellulose, around 99%, suggests that it can be used as a hydrogel, which is known for its favorable biocompatible properties and lack of protein adsorption. Its physical properties make it extremely attractive as an implant for biomedical applications such as cartilage replacement, vascular grafts (Svensson, A., Nicklasson, E. Harrah, T., Panilaitis, B., Kaplan, D. Brittberg. M, and Gatenholm, P., 2005, Bacterial Cellulose as a Potential Scaffold for Tissue Engineering of Cartilage, Biomaterials, 26, 419-431; Klemm, D; March S., Schuman, D., et al. 2001, Method and device for producing shaped microbial cellulose for use as biomaterial, especially for microsurgery WO2001061026), or as a hydrophilic coating of other biomaterials. Different fermentation conditions can also affect the morphology of bacterial cellulose. For example, agitation plays a very important role for the production of cellulose. Acetobacter xylinum is rather difficult to culture in traditional fermentation technology. During agitation bacteria can switch off cellulose production. However, the culture, when subjected to gentle shaking, has been shown to produce a much looser network. The shape and morphology of BC material can also be controlled by oxygen delivery at the nutrient-air interface. This has been particularly useful for developing the technology platform for the preparation of BC tubes using submerged cells on an oxygen-permeable support together with a gas inlet through the support (Bodin, A., Bäckdahl, H., Fink, H., Gustafsson, L., Risberg, B., and Gatenholm, P., Influence of cultivation conditions on the mechanical and morphological properties of bacterial cellulose tubes, Biotechnology and Bioengineering, 2007, 97 (2), 425-434).

In particular, oxygen delivery at the media-air interface has been shown to enhance cellulose production resulting in a denser cellulose network especially on the inner wall of the tube. High oxygen consumption at the interface results in a highly anisotropic fiber network. By contrast, a very open nanofibril structure is produced on the outside of the BC tube when cultured with an oxygen tension of 100%. At lower oxygen concentrations, the tubes will have less density at the inner surface, a less porous outer nanofiber network, and less anisotropy. These tubes have been used successfully as in vivo replacement materials for vasculature.

In addition to BC tube implants as vein or arterial replacement, biocompatibility of BC has also been validated in subcutaneous implants in rats for 1, 4 and 12 weeks. There were no macroscopic signs of inflammation, such as redness or exudates around the implanted BC pieces or in the incision at any time point. Overall, there were no histological signs of inflammation in the specimens, i.e. an abnormally high number of small cells in the connective tissue and especially around the blood vessels in the connective tissue (Helenius, G., Bäckdahl, H., Bodin, A., Nanmark, U., Gatenholm, P., and Risberg, B., 2006, In vivo Biocompatibility of Bacterial Cellulose, J. Biomed. Mater. Res. A., 76(2), 431-438.

All these observations taken together suggested that BC is very attractive as a biomaterial and particularly as a scaffold for tissue engineering.

BC holds particular promise as a potential meniscus implant (Bodin, A., Concaro, S., Brittberg, M., and Gatenholm, P., 2007, Bacterial Cellulose as a Potential Meniscus Implant, Journal of Tissue Engineering and Regenerative Medicine, 48, 7623-7631). Naturally-occurring (and healthy) meniscus has a number of mechanical properties that provide cushioning and axial load-bearing by supporting resultant tensile hoop stresses during movement of the knee and allows the joint to bear weight of the individual while standing and walking. These mechanical properties are however lacking in BC that was randomly deposited within a form designed to duplicate the macrostructure of a natural meniscus. The random nature of the nanofibrils within BC limits its usefulness, since applications like meniscus, tendons, ligaments, heart valves, cartilage require specific characteristics outside the parameters inherent in the natural material, particularly those with precise ranges of mechanical performance. Collagen fibrils are for example oriented predominantly in the circumferential direction which make meniscus much stiffer in this direction (Skaggs, D. L., Warden, W. H., and Mow, V. C., 1994, Journal of Orthopaedic Research, 12, 176-185).

The ability of mimetic fibrils to initiate growth of crystals such as hydroxyapatite is an attractive way to promote cell adhesion and differentiation. A composite biocompatible hydrogel material consisting of bacterial cellulose and calcium salts has been suggested for use as bone graft material (Hutchens, S. A., et al, 2004, US2004/0096509A1. Unfortunately, despite their nanoscale porosity, such materials do not allow cells to migrate into the structure. The failure of this simple mimetic is partly due to its relatively tight structural network of cellulose nanofibrils. Introduction of micro- and macroporosity in a precise and controlled manner could create pores that would be appropriate for cell migration and might also promote cell-cell interactions that are required for recapitulation of complex organ structures.

There remains an unmet need in the field to develop methods of fabricating biocompatible materials with controlled architecture on different length scales (e.g. nano, micro and macro) controlled microporosity and controlled mechanical and chemical properties. One approach that has not yet been exploited is the use of electrical or magnetic fields to control motion of cells which produce biopolymers.

The study of cells in response to electric fields has been studied extensively for decades. In particular the motion of cells under an applied electric field has been studied for several decades, as has their response to uniform and non-uniform electric fields. For example, dielectrophoresis (DEP) is the motion of a particle due to its polarization induced by the presence of a non-uniform electric field. It has been shown that DEP can be used to transport suspended particles utilizing either oscillating (AC) or steady (DC) electric fields. DEP is suitable for differentiating biological particles (e.g., cells, spores, viruses, DNA) because it can collect specific types of particles rapidly and reversibly based on intrinsic properties including size, shape, conductivity and polarizability. Many device architectures and configurations have been developed to sort a broad range of biological particles by DEP. For example, early DEP experiments carried out by Pohl, H. A. (Pohl, H. A., 1978. Dielectrophoresis the behavior of neutral matter in nonuniform electric fields. Cambridge University Press, Cambridge) utilized pin-plate and pin-pin electrodes to differentiate between live and dead yeast cells and collected them at the surface of the electrode. Typical dielectrophoretic devices employ an array of thin-film interdigitated electrodes placed within a flow channel to generate a nonuniform electric field that interacts with particles near the surface of the electrode array. The nonuniform electric fields are typically generated by a single-phase AC source, and in addition, multiple-phase sources can trap and sequentially transport particles in a technique called traveling-wave dielectrophoresis. Another approach is insulator-based dielectrophoresis (iDEP), which uses insulating obstacles, instead of electrodes, to produce spatial nonuniformities in an electric field that is applied through the suspending liquid. DEP platforms have shown that DEP is an effective means to manipulate and differentiate cells based on their size, shape, internal structure, and intrinsic properties such as conductivity and polarity. None of above mentioned methods have been used for control of motion of cells with simultaneous production of extracellular polymers and using an electromagnetic field for controlling the biofabrication process.

### SUMMARY

The present invention provides devices and methods to direct the movement of biopolymer-producing cells in order to produce biopolymer networks with defined architectures and dimensions. In one embodiment, the cells are nanocellulose- producing bacteria which, as they move through a liquid media, leave behind a “trail” or “thread” of extruded cellulose nanofibrils. According to the invention, the position of the extruded cellulose can be varied by controlling the three-dimensional movement of the bacteria through the surrounding medium, e.g. by manipulating electromagnetic fields which are applied to the medium. This invention increases biopolymer production by increasing the oxygen concentration within the media through electrolysis.

It should be noted that polymer production can be halted by applying a field such as that which is induced by irreversible electroporation. This invention can additionally deposit ions onto the biopolymer by incorporating free ions into the media while applying an electromagnetic field. As a result, a variety of mimetic biocompatible materials of any desired architecture can be produced for use, for example, as implants, for tissue replacement and/or regeneration, etc.

The present invention provides a method of producing a predetermined pattern of ordered biopolymers. The method comprises the steps of 1) providing biopolymer-extruding cells in a liquid medium under conditions suitable for extrusion of biopolymers into said liquid medium by said biopolymer-extruding cells; and 2) applying an electromagnetic field to the liquid medium in a manner that causes the biopolymer-extruding cells to move according to the predetermined pattern while extruding the biopolymers, thereby forming the predetermined pattern of ordered biopolymers. In some embodiments, the method further comprises the step of varying the electromagnetic field. The predetermined pattern that is formed may be three-dimensional, and the method may also comprise the step of generating the electromagnetic field by suspending electrodes in the liquid medium. In one embodiment, the electrodes are operated in a manner which produces oxygen. In another embodiment, the electrodes are operated in a manner which produces ions from media components. In some embodiments, movement of the biopolymer-extruding cells in the applied electromagnetic field (i.e. in the liquid media in response to the applied electromagnetic field) is unidirectional, while in other embodiments, movement is multidirection, e.g. bidirectional, tridirectional, etc. The method may further comprise the step of halting extrusion of the bioplymers by the bacteria, for example, by subjecting the biopolymer-extruding cells to an applied electric field sufficient to induce death. In some embodiments, in order to halt production and/or extrusion of the biopolymer by the cells, an electric field sufficient to induce a 1V or greater drop in potential across a cell membrane is applied, thereby inducing irreversible electroporation of the cells. In some embodiments, in order to halt or cease biopolymer production, an electric field sufficient to lyse the biopolymer-extruding cells is applied. In some embodiments of the invention, the movement of the biopolymer-extruding cells in the applied electromagnetic field traces a curve, and hence extrusion and deposition of the biopolymers is in the shape of a curve (i.e. is an arc, is elliptical, is a loop, is sinusoidal, etc.). In some embodiments, the predetermined pattern of ordered biopolymers forms at or near a gas-liquid interface of said liquid medium, e.g. at locations or in areas near the interface where sufficient oxygen is present to support physiological activity of the cells. In some embodiments, the biopolymer-extruding cells are bacterial cells, for example, of a as species selected from Acetobacter, Agrobacterium, Rhizobium, Pseudomonas and Alcaligenes. In some embodiments, the bacteria are Acetobacter xylinum or Acetobacter pasteurianus. In some embodiments of the invention, the biopolymers are bacterial cellulose. In some embodiments of the invention, the electromagnetic field is an electric field. In some embodiments, the electric field may range from about 0.1 V/cm to about 100V/cm or greater. In some embodiments of the invention, the step of varying the electromagnetic field is carried out by a programmed computer. In some embodiments, the predetermined pattern includes pores. The pores may be of a size sufficient to allow infiltration (e.g. entry, passage through, etc.) of animal or human cells into the pores.

The invention also provides a device for producing a predetermined pattern of ordered biopolymers. The device comprises: 1) a container for containing biopolymer-extruding cells in a liquid medium under conditions suitable for extrusion of biopolymers into the liquid medium by the biopolymer-extruding cells; and 2) means for applying an electromagnetic field to said liquid medium in a manner that causes the biopolymer-extruding cells to move according to the predetermined pattern while extruding the biopolymers, thereby forming the predetermined pattern of ordered biopolymers. The application of the electromagnetic field may be carried out by a computer programmed to do so.

The invention also provides a method of forming a predetermined pattern of ordered biopolymers. The method comprises the steps of 1) providing biopolymer-extruding cells in a liquid medium under conditions suitable for extrusion of biopolymers in liquid at or near a liquid-oxygen interface, by the biopolymer-extruding cells; 2) suspending electrodes in the liquid medium; and 3) operating the electrodes in a manner which generates one or more liquid-oxygen interfaces in the liquid media, whereupon said biopolymer-extruding cells extrude the bioplymers in the liquid at or near the one or more oxygen-liquid interfaces in the predetermined pattern of ordered biopolymers. The liquid-oxygen interfaces may be bubbles.

The invention also provides a device for producing a predetermined pattern of ordered biopolymers in vitro. The device comprises 1) a container for containing biopolymer-extruding cells in a liquid medium under conditions suitable for extrusion of biopolymers in liquid at or near a liquid-oxygen interface, by the biopolymer-extruding cells; and 2) means for generating one or more liquid-oxygen interfaces in the liquid media in a manner that causes the biopolymer-extruding cells to extrude the bioplymers in the liquid at or near the one or more oxygen-liquid interfaces in the predetermined pattern of ordered biopolymers.

The invention further provides a medical implant, comprising a polymeric material at least a portion of which includes a predetermined pattern of ordered biopolymers including one or more fibrils oriented in a manner which provides a specified tensile strength in at least one dimension. The medical implant may further comprise at least one opening which passes through the polymeric material. In addition, in some embodiments, the polymeric material is configured in a form of a human meniscus or other cartilage tissues. For example, the polymeric material may be configured in a form suitable for a bone graft, or may be configured in a form of tendons or ligaments, or in a form for neural network support.

The invention further provides a polymeric material at least a portion of which includes a predetermined pattern of ordered biopolymers including one or more fibrils oriented in a manner which provides a specified tensile strength in at least one dimension. In some embodiments, the predetermined pattern is in the fowl of a weave.

The invention also provides a multilayered polymeric material including a plurality of layers each of which includes at least one predetermined pattern of ordered biopolymers including one or more fibrils oriented in a manner which provides a specified tensile strength in at least one dimension.

The invention also provides a scaffold for tissue engineering, cell differentiation and organ regeneration. The scaffold comprises a polymeric material at least a portion of which includes a predetermined pattern of ordered biopolymers including one or more fibrils oriented in a manner which provides a specified tensile strength in at least one dimension and comprising at least one opening which passes through the polymeric material.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 A shows schematic layout of the process for fabrication of the device and FIG. 1 B dimensions of the device, FIG. 1C the device in use and FIG. 1D shows motion of bacteria in the device.

FIG. 2 A shows the motion of bacteria in the electric field and FIG. 2B and 2C are Scanning Electron Micrographs showing aligned scaffolds based on bacterial cellulose.

FIG. 3 A shows tensile testing data (load versus elongation) on aligned BC fibrils compared with random BC network and FIG. 3B shows modulus of aligned BC compared with random BC.

FIG. 4 shows isometric, top, and side schematics of 10 mm×10 mm PDMS device used to demonstrate bidirectional motion of bacteria. The device contains 40 inlet ports, 9 on each edge and one at each corner, into which cells or bacteria can be injected and voltages applied via electrodes.

FIG. 5 shows simulated electric field lines inside the channel with (A) voltages decreasing 5%/inlet from 100-50% along top and keft side and from 50-5% along the bottom and right side, Ground is applied at the bottom bright corner inlet and (B) 100% applied to each port along the top and left edges, ground applied at the bottom right corner port, and remaining ports left floating.

FIG. 6 illustrates schematically devices for manufacturing of desired (A) zigzag and (B) cross hatch patterns.

FIG. 7 shows on the left side schematic side view of multi layer BC growth with (A) initial 250 mL of growth media, (B) first BC scaffold layer, (C) two sequential BC growth layers, and (D) four sequential BC growth layers. The figure on the right side (E) shows how electric fields modify cellulose production: The formation of bacterial cellulose below the liquid-air boundary is amplified due to the oxygen rich environment created by the electrolysis of water. This allows for greater flexibility in device design.

FIG. 8 shows simulated electric field lines inside the channel with injected porogen particles.

FIG. 9 shows the effect of oxygen at 1 V applied formed due to electrolyses on the production of 3D structure.

FIGS. 10a and b shows the manufacturing of highly porous material using combination of oxygen delivery through electrolyses combined with the effect of bubbles which act as porogens. FIG. 10a shows actual bacterial cellulose; FIG. 10b shows a schematic of a device to carry out this embodiment of the invention.

FIG. 11 A shows the schematic layout of process for electromagnetic manufacturing of oriented fibrils in the circumferential directions for mimetic biocompatible meniscus implant. FIG. 11B shows the sheep meniscus which is used as the animal model for evaluation of BC meniscus.

FIG. 12 shows a multi layer scaffold with different predetermined layers/patterns.

FIG. 13 shows a system level schematic of the microweaver.

FIG. 14 shows an example of a chamber with integrated porosity and insulating bathers to create multiple layers of BC with prescribed fiber orientations. a) Top View b) ISO 3D view c) Side View.

FIG. 15 shows an example of a chamber that uses DEP and electrophoretic forces to create complex fiber orientations.

FIG. 16 shows an example of a chamber capable of halting biopolymer production by inducing irresistible electroporation.

FIG. 17 shows a flow chart of polymer production.

FIG. 18 shows an example of a field cage production chamber with individually addressable electrodes.

FIG. 19 shows an example of chamber capable of producing scaffolds with multiple different fiber orientations.

### DETAILED DESCRIPTION

This invention describes a novel technology platform in which an electromagnetic field is used to control the biofabrication of mimetic biocompatible materials. The materials may be used as scaffolds in tissue engineering and regenerative medicine, in biomedical devices, as biocompatible coatings, and in many other health care products. According to the invention, an electromagnetic field is used to control the motion of cells which produce biopolymers capable of assembling (or being assembled) into useful nanofibers, in order to biofabricate a wide range of material architectures. For example, in one embodiment, cellulose nano-fibrils are produced by bacteria. The methods and devices of the invention also enable the introduction into the biofabricated material, of micro- and macroporosity, as well as the deposition of ions or other substances of interest onto the biopolymers. In addition, the mechanical and chemical properties of the biofabricated materials can be controlled. Since the materials are made from natural biopolymers (e.g. collagen), they are highly biocompatible, i.e. they are unlikely to elicit an immune response or to be rejected by a recipient.

The invention thus solves one of major limitations in tissue engineering and regenerative medicine, biomedical devices and health care products, namely control of architecture and morphology of biomaterials. The invention is based on the discovery that electromagnetic devices can be used to control the motion of cells, such as bacteria Acetobacter xylinum cells, in multiple directions with simultaneous production of an oriented biopolymer, such as nanocellulose. In one embodiment, the controlled production of bacterial cellulose at the nanoscale level was accomplished by cellulose deposition during unidirectional motion of bacteria in an electric field, and during oscillatory and reversing motion within an electric field. Using the methods of the invention, layers of cellulose can be assembled into any desired two- or three-dimensional shape. In addition, the structures may include porogens which provide microporous structure. Computer aided guidance of the applied electromagnetic field allows fabrication of a three-dimensional network with good mechanical properties, with tailor-made chemical properties, with the ability to support a micro-scale fluid flow, and with the ability to allow cells to attach to and enter the structures. Significantly, in some embodiments (e.g. collagen), the biopolymers in the materials that are fabricated are aligned in the field in a manner that results their association into hierarchically organized (aligned) nanofibrils. These nanofibrils display increased tensile strength, compared to randomly deposited polymers, and, even though they are fabricated in vitro, their properties thus mimic those of the extracellular matrix of human or animal tissues formed in vivo.

### Definitions

Medium: any liquid or gel capable of sustaining cells for a period of time.
Ordered polymer or polymers: polymers which are not amorphous; it is semicrystalline or highly crystalline, such polymers will assembly in the most cases into nanofibrils, microfibrils or fibers.
Biopolymers: polymers produced by biological organisms.
Predetermined pattern: a pattern which determined before the process and is achieved by means of controllinga direction of movement;
Move or movement motion of the cell relative to the medium (e.g., electrophoretically) or relative to the device (electro-osmosis).
Varying: used in the context of varying the electric field, the magnitude and/or the frequency is adjusted after periods of time and different sets of electrodes can be energized.
Fibrils: molecules assembled into bundles which have aspect ratio (length divided by diameter) higher than 5 specified tensile strength; strength of material determined by tensile test oriented;
Orientated: directed along a path due to the electrical forces that the particle and media are subjected.
Weave: biopolymers interwoven due to natural growth or due to the applied field.
Three-dimensional: stacking of layers or fabricating a layer with substantial thickness.
Unidirectional: directed along a potential or field gradient (or some superposition) in a primary direction.
Bidirectional: multiple varying directions in series or a superposition of forces such that motion of the bacteria is induced in more than one dimension.
Porogen: particles or processes that generate openings or pores in a material.
Openings and pores: architecture of material in which discontinuity occurs.

Herein, the terms “biopolymer” and “polymer” may be used interchangeably, and both refer to the extruded material (usually but not always a polymeric string or chain of chemically linked monomeric units) that is produced by a cell (e.g. a living cell), as described herein. A plurality of biopolymers, when aggregated together, may be referred to as a “fiber” or “nanofiber” or “fibril” or “nanofibril”, or by other similar terms. Generally, “fibrils” refer to bundles of molecules which are assembled into assemblies which have aspect ratios (length divided by diameter) higher than about 5.

The electromagnetic biofabrication processes and devices of the invention employ at least the following components:

1) cells which are capable of synthesizing one or more extracellular biopolymers of interest;
2) media in which the cells can be suitably maintained under conditions conducive to the bioproduction of the one or more extracellular polymers of interest, and which is susceptible or amenable to the application of an electromagnetic force;
3) at least one source of electromagnetic force; and
4) a container or device for containing the cells and the media, in a manner that allows the application of electromagnetic force to the cells in the media.
Each of these components is discussed in detail below.
CELLS AND THE POLYMERS THEY EXTRUDE: The cells that are employed in the practice of the invention may be of any cell type that is capable of synthesizing a biopolymer of interest. The cell must be capable of synthesizing the biopolymer and of extruding the biopolymer into the surrounding media in a manner that produces a substantially continuous biopolymer thread or fibril in its wake as it moves through the medium. The cells that are utilized in the invention are capable of movement, either on their own (i.e. they are motile cells which use energy to move spontaneously and actively) or in response to an applied electromagnetic force, i.e. movement is caused by imposition of an electromagnetic field and the cell does not expend energy to move or both. If the cells are motile and can “swim” through the medium without any added stimulus, they must be amenable to being induced to move in a particular direction in response to an applied electromagnetic force. The cells may be prokaryotic; as used herein “prokaryotic” encompasses both bacteria and archaea. Alternatively, the cells may be eukaryotic. In the latter case, in some embodiments, the cells are removed from a multicellular organism and/or obtained from a cultured cell line or other source, and suspended in medium in order to carry out the biofabrication process. However, this need not always be the case. In some embodiments, the eukaryotic organisms are small enough to be cultured and maintained by being suspended in a liquid environment, and lightweight enough for their movement to be manipulated by an electromagnetic field while in a liquid medium. Examples of eukaryotic cells include but are not limited to ex vivo cells originating from (i.e. originally removed from) complex animals such as mammals (e.g. humans or other mammals) or other animals; fungi; slime molds; algae; and protozoa. Further, while in most embodiments, the cells used in the invention are in the form of single cells, this need not always be the case. In some embodiments, various aggregates of cells (e.g. clumps, strings, sheets, etc.) may he used to advantage, or at least may be used without causing a disadvantage.

The extracellular biopolymers that are synthesized or produced by the cells that are employed in the invention include but are not limited to collagen, elastin, fibrin, silk, keratin, tubulin, actin, cellulose, xylan, chitin, chitosan, glycosaminoglycans, hyaluronic acid, agarose, alginate, etc. In some embodiments, the cells have a native or natural capacity to produce these materials. In other embodiments, the cells can be genetically modified to produce one or more biopolymers, or to alter the properties of the biopolymer (e.g. the composition, tensile strength, dimensions, crystallinity, moisture sorption, electrical properties, magnetic properties, acoustic properties, etc.) or the capacity of the cell to produce the biopolymer may be altered (e.g. to produce larger quantities, or to use diverse energy sources or substrates to produce the biopolymer, or to produce the biopolymer in response to cues such as changes in temperature, pH, media composition, oxygen concentration, light, pressure, electromagnetic field, etc.) In addition, the cells may be genetically engineered to control other useful properties, including but not limited to their charge; the ability to produce a biopolymer if they do not naturally do so; the ability to produce more than one bioplymer, e.g. to produce one or more biopolymers in addition to those that they naturally produce.

The cellulose producing bacteria may be Acetobacter, Agrobacterium, Rhizobium, Pseudomonas or Alcaligenes most preferably species of Acetobacter xylinum or Acetobacter pasteurianus. The most preferred strain is Acetobacter xylinum subsp.sucrofermentas BPR2001, trade number 700178™, from the ATCC.

Another type of cells can be animal or human fibroblasts producing collagen, elastin and proteoglycans. Example is NIH3T3 (designation refers to the abbreviation of “3-day transfer, inoculum 3×10⁵ cells). This cell line was originally established from the primary mouse embryonic fibroblast cells. Animal or human stem cells can also be used.

Further, the cells may be modified by other non-genetic means to enhance their usefulness in the practice of the invention, such modifications including but not limited to: binding magnetic or conducting nanoparticles or polymers to enhance their charge; or introducing into the cell magnetic or conducting nanoparticles or quantum dots into the cell.

With respect to extrusion of the biopolymer, the cells themselves are in liquid media when the biopolymers are extruded, and the biopolymers are generally extruded into the liquid media. In some cases, extrusion occurs at or near a liquid-gas interface (e.g. at the interface of medium and air or oxygen), since the cells may be of a type that require oxygen to survive and/or to produce the biopolymer, e.g. extrusion of cellulose by Acetobacter species. In such cases, the biopolymers generally aggregate after extrusion and the aggregate may be partially present in the liquid medium and partly protruding from the medium into the air, i.e. the aggregate may “float” or appear to float on the surface of the medium. Thus, herein, when extrusion “at” or “near” a gas-liquid interface is referred to, we mean that extrusion generally takes place in the medium but in the vicinity of the gas phase, e.g. within about 0.5 to 5 cm of the gas phase, where the medium is sufficiently oxygenated. Such liquid-gas interfaces may but do not always occur at the “top” of the medium; they may also be generated at any point throughout the volume of the medium, e.g. by bubbles of, for example, air or oxygen, as described in detail below.

Generally, once extruded, the individual polymers aggregate to form larger structures of ordered biopolymers. By “ordered bioplymers” we mean a plurality of polymers that are not arranged amorphously with respect to each other. Rather, the ordered polymers are semicrystalline or crystalline. Such ordered arrangements of polymers may take the form of, for example, fibers or fibrils, which may in turn be arranged in larger, non-random structures, e.g. layered structures (described in detail below), or structures that are deposited in or on a template or mold and thus take on the shape of the template/mold, etc. In this case, ordered refers to both the nano- and/or micro-structure of the polymers, and to the macrostructure of material made from the ordered polymers. Further, while in most embodiments of the invention, the polymers are ordered, this need not always be the case. In some embodiments, some portions of a structure or material of the invention may disordered or amorphous, i.e. the invention encompasses materials and structures of which at least a portion is amorphous or disordered (not semicrystalline or crystalline). The ordering of the polymers may be the result, for example, of hydrogen bonding, van der Walls interactions, ionic interactions (attraction or repulsion), and in some cases, of covalent bonding.

In some embodiments of the invention, a single cell type is used to prepare a single type of biopolymer, and hence homogenous fibers are formed. However, this need not always be the case. In some embodiments, different types of cells may be mixed together in the medium to produce composite materials, e.g. the polymers produced by one of the types of cells will be mixed or aggregated with polymers produced by another type of cell. For example, two different types of polymers may be produced and extruded by two different cell types in close proximity to each other in the medium, and the polymers may form a composite fibril or composite random structure. Alternatively, homogenous fibers may be formed by each polymer, and the homogenous fibers may aggregate to form a composite “mat” or “net” of material containing multiple types of fibers. Alternatively, one cell may be capable of producing more than one type of biopolymer.

MEDIUM: The medium in which the cells are maintained during biopolymer production may be any of many suitable types. The medium is generally liquid, and of a viscosity that allows the cells to move or be moved through the medium in response to directional prompting by application of an electromagnetic field. The viscosity of the medium may be altered to produce desired speeds of movement or patterns of distribution of the cells. Further, in some embodiments, the medium may be a gel. In this embodiment, the movement of the cells may be somewhat curtailed, but the imposed electromagnetic field is still capable of eliciting movement such as orientation of the cells, spinning in place, etc. Deposition of polymers in gels may produce more tightly packed polymer formations.

Those of skill in the art are generally familiar with the culture of cells in liquid suspension. Such cultures are usually aqueous, and contain various nutrients and supplements that permit growth and/or maintenance and metabolic activity of the cells, and are suitably oxygenated or not, depending on the requirements of the cells. For the practice of the present invention, the general requirements are that the medium must sustain the cells in a manner that: 1) allows the cells to produce the biopolymer(s) of interest; and 2) allows transmission of an applied electromagnetic force to the cells in the medium in a mariner that permits the cells to respond to the force in a desired manner. The nutritive components of the medium may be used by the cell for general metabolic and catabolic activities, as well as to build the biopolymer(s) of interest. Further, the medium may be supplemented in particular to support biopolymer synthesis (e.g. by providing an abundant source of e.g. monomeric polymer building blocks, or to bias the cellular metabolism in favor of biopolymer synthesis, etc.). Examples of suitable media for growing bacteria include but are not limited to: Schramm-Hestrin-medium which contains, per liter distilled water, 20 g of glucose, 5 g of bactopeptone, 5 g of yeast extract, 3.4 g of disodium-hydrogenphosphate dehydrate and 1.15 g of citric acid monohydrate and which exhibits a pH value between 6.0 and 6.3; 0.3 wt % green tea powder and 5wt % sucrose with pH adjusted to 4.5 with acetic acid; Medium composed of (fructose [4% w/vl], yeast extract [0.5% w/v], (NH₄)₂SO₄ [0.33% w/v], KH₂PO₄ [0.1% w/v], MgSO₄.7H₂O [0.025% w/v], corn steep liquor [2% v/v], trace metal solution [1% v/v, (30 mg EDTA, 14.7 mg CaCl₂.2H₂O, 3.6 mg FeSO₄.7H₂O, 2.42 mg Na₂MoO₄.2H₂O, 1.73 mg ZnSO₄.7H₂O, 1.39 mg MnSO₄.5H₂O and 0.05 mg CuSO₄.5H₂O in 1 liter distilled water)] and vitamin solution [1% v/v (2 mg inositol, 0.4 mg pyridoxine HCl, 0.4 mg niacin, 0.4 mg thiamine HCl, 0.2 mg para-aminobenzoic acid, 0.2 mg D-panthothenic acid calcium, 0.2 mg riboflavin, 0.0002 mg folic acid and 0.0002 mg D-biotin in 1 liter distilled water)]). Any medium comprised of sugar source, nitrogen source and vitamins can be successful used. Bacteria grow even in apple or pineapple juice, coconut milk, beer waste, or wine.

Media to grow mammalian cells is typically composed of glucose, growth factors and other nutrients. The growth factors used to supplement media are often derived from animal blood such as calf serum.

The media may be altered to include ions such that ions are deposited onto the biopolymer. This can include but are not limited to: Schramm-Hestrin-medium with 1, 5, or 10% PBS (Phosphate Buffered Saline), Schramm-Hestrin-medium with 1%, 5%, or 10% 0.1 molar calcium chloride, or any suitable culture media with an increased concentration of one or more ions. Ions may include but are not limited to potassium, calcium, phosphate, or sodium. These media are easily created by those skilled in the art.

In addition, the composition of the media used in the practice of the invention should be commensurate with the application of an electromagnetic field, and transmission of the field to the cells. The type of buffer is essentially dependent on what is necessary to have the right properties to keep cell viable. Typically, experiments are conducted in deionized water, phosphate buffered solution, culture media.

Any of several methods may be used to stop the polymer extrusion process, including but not limited to addition of a substance that is lethal to the cells, application of heat or cold sufficient to kill the cells (e.g. boiling, freezing, freeze-drying, etc.), or by manipulating the electromagnetic field, e.g. by irreversible electroporation, as described below.

ELECTROMAGNETIC FIELD: Directed, controlled or guided deposition of biopolymers by cells according to the invention is accomplished by exposing the cells to an electromagnetic field in order to guide their movement or position within the medium. The electromagnetic field may control the motion/movement of the cells, or of the material they produce, or both. The electromagnetic field may be 1) electric field; 2) a magnetic field; or 3) a combination of both, i.e. an electric field in combination with a magnetic field. If an electric field is used, the voltage will generally be, for example, a direct current voltage in the range of 0.001V to 5000V (typically between 0.1 V and 50V). Such electrical currents may be imposed on the medium containing the bio-polymer producing cells by any of several means, including but not limited to: by positioning or including electrodes in or outside the device of the invention (which is described in detail below); or by a contactless electrode method in which capacitive dielectric barriers isolate the electrodes from the culture media. The AC electric voltage is generally in the range of from about 0.00001 V to about 5000 V, and is preferably in the range of from about 0.001 V to about 50 V. The exact voltage that is applied will vary from circumstance to circumstance, and may depend on the type of cells being used, the medium, the biopolymer being produced, the electrode geometry, chamber configuration, electromagnetic properties of the cells, device, or biopolymer being synthesized, and the desired characteristics of the object being synthesized and may any value up to which the electric field induces lysis, irreversible electroporation, boiling, or unwanted cell death. Typically the voltages applied will be sufficient to induce an electric field generally in the range of from about 0.01 V/cm to about 1000 V/cm, and is preferably in the range of from about 0.1 V/cm to about 100 V/cm.

In some embodiments of the invention, a magnetic field is used to manipulate the movement and/or positioning of the cells. In yet other embodiments, a combination of electric and magnetic fields is utilized. In all embodiments, the applied field may be constant throughout the deposition process, or may be varied during the process so as to achieve a desired result. For example, the applied field may be used to change the direction of flow (movement of the bacteria, and hence the position of the polymers when extruded). The applied field can be used to direct the cells via dielectrophoresis, traveling wave dielectrophoresis, magnetophoresis, electroosmosis, electrophoresis, thermophoresis, AC electroosmosis and the like and in superposition. It also should be noted that the fields may be either AC or DC or both (e.g. an AC field with a DC offset). It also should be noted that more than one field can be applied simultaneously or in sequence. For example, the cells can be directed using electrophoresis with a DC field for a period of time and then redirected using dielectrophoresis with an AC field.

In addition, cellulose production can be halted by killing the cell using the field. The field can be applied such that a voltage across the membrane is sufficient to induce irreversible electroporation. This voltage is on the order of 0.5-5V. Furthermore, the fields can induce cell death via electrical or thermal lysis. In addition, the cells can be left viable but moved too quickly through an area to deposit a biopolymer.

DEVICES: According to the invention, devices are provided which include elements necessary to carry out the invention. Two general types of devices are encompassed, although the invention is not limited to these.

In one embodiment, referred to as a “microweaver”, the device is one in which cells and a nutrient solution (e.g. media) are placed, and in which electrodes provide an electrical current. The device may be of a type including but not limited to: a microfluidic device; a shallow plate-like device for producing largely two-dimensional materials (e.g. materials that are in the shape of mats or sheets of a desired length and width, and which also will be of a desired depth); or a container of a shape and volume which allows for production of three dimensional materials, e.g. materials with more complex features, such as spherical or curved portions, etc. In a second embodiment, the device is one in which different biopolymeric objects or materials made according to the practice of the invention, are further modified by joining in “zigzag” or “cross hatch” patterns, using cells controlled by electromagnetic field. This embodiment of the device is referred to herein as a “micro-sewing machine”.

This can be accomplished by applying a low-frequency AC field using two skewed electrodes. The electric field will drive the cells horizontally via dielectrophoresis while they ‘oscillate’ electrophoretically due to the AC field. Also, electrolysis can be accomplished (or suppressed) with either an AC field or a DC.

In all embodiments of the invention, the application and variations of the applied electromagnetic field may be controlled by a computer programmed to do so. The invention thus also provides software comprising instructions for causing a computer to carry out a program which guides the production and application of an electromagnetic field to the device. A computer or computerized system to carry out the methods of the invention may include, for example, stand alone electronics, microprocessors, and oscillating crystal devices, etc. The electromagnetic field is of strength sufficient to elicit movement of cells in the medium within the device, in a manner that results in deposition of biopolymers by the cells in a desired pattern.

The final shape of the material or object that is fabricated according to the invention is the result, at least in part, of manipulation of the electromagnetic filed to which the cells and the incipient biopolymer are exposed during fabrication. The precise procedure for attaining the desired shape will vary according to the cell type that is used and the biopolymer that is produced. For example, when bacteria are used to generate nanocellulose, they do so only at or near a liquid-gas interface, i.e. at or near the point or points of contact between the liquid medium in which they are suspended and a gas (e.g. oxygen) or mixture of gases (e.g. air). In some embodiments, the biopolymers and fibrils that are produced are thus in the form of a sheet on the surface of the medium. Herein, such a sheet may be described as “2-dimensional” in that it is comprised of a single layer of e.g. nanocellulose fibrils, the surface of which has a defined area that can be expressed as square units, e.g. mm² or cm². Such a two-dimensional material has a high surface to volume ratio, and can be converted into a “three dimensional” shape by any of several methods, e.g. by forming multilayer structures. For example, an initial or first layer is formed, the first layer is covered with media and a second layer is formed over the first layer, and so one. By repeating this process, multiple subsequent layers are formed, e.g. from about 2 to about 10,000 or more layers may be formed. When a desired number of layers have been formed (i.e. when a desired thickness has been attained, or at some other point in layer formation), media retained between the layers is removed and a solid, 3-dimensional structure results. Variations of overall shape of the material can be made by, for example, varying the shape of the substrate (template, mold, etc.) on or in which the bacteria (or other cells) deposit the polymers. For example, deposition may occur in a channel or trench, or in a circular depression, or in any desired shape. Further, the deposited material may be mechanically trimmed to any desired shaped following fabrication.

In addition, the position of the liquid-gas interface may be changed by various means. For example, air or oxygen may be bubbled through the medium, and or the viscosity of the medium and/or the bubble size may modulated so that bubbles remain suspended in the media. Bacteria suspended in the medium are able to manufacture collagen at (near) the multiple liquid-gas interfaces provided by the bubbles. Alternatively, oxygen bubbles may be advantageously introduced due to electrolysis of water, as a result of electrodes in the device that are used to produce an electric field to control the movement of cells.

The orientation of the polymers within the material of the invention may be advantageously varied by varying the electromagnetic field to which the cells are exposed during polymer extrusion. For example, the force, location and/or timing of the field may be varied during the extrusion/deposition process. As a result, the position and/or movement of the cells within the medium also varies, e.g. so as to cause the cells to move in a straight line, to turn, to “zig-zag”, to oscillate, etc. through the medium. Polymer extrusion occurs wherever a cell is located and thus predetermined patterns of polymer extrusion or deposition can be designed and implemented by variations in the electromagnetic field. By “predetermined pattern” we mean a pattern that is planned, decided upon or determined in advance, and that is not random. As used herein, a “predetermined pattern” of polymer extrusion or deposition correlates with or results from planned, non-random variations in the electromagnetic field which is applied to the cells that are producing biopolymers. Such variations in the EM field cause variations in where or possibly how the cells move within the medium, and include controlling or influencing: the direction of movement, i.e. the trajectory of the cells in the medium; the speed of movement; the shape of a path traced by the movement of the cells; holding cells stationary; when holding cells stationary, influencing nanoscale movements such as twirling, oscillating, rotating, etc. Movement of the cells in the field may be unidirectional, bidirectional, or multidirectional, depending on the desired predetermined pattern.

In some embodiments of the invention, the field is held constant so that the polymers themselves align or orient with the field. It has been determined that nanoscale alignment or orientation of polymers (for example, collagen polymers) in this manner results in the production of collagen fibrils with improved tensile strength, as described in the Examples below. By “tensile strength” we mean the strength of material as determined or measured using a tensile test, as is known in the art. This process enables production of biocompatible materials composed of organized, ordered, aligned nanofibrils which have better mechanical properties than random networks. This is particularly important in applications such as implants and scaffolds for ligaments, tendons, meniscus, hearts valves and bone. The material which is composed of aligned nanofibrils enables animal or human cell orientation which is crucial for regeneration of tissues such as nerves and building of muscles. This process enables layer by layer production of 2D oriented layers which can be assembled into 3D objects, as described above.

Other modifications to the process may also be made. For example, it is highly desirable to create micro-porous biomaterials that, for example, allow cells to migrate into and through the materials via the pores. Pores include channels, holes, openings, and other discontinuities in the structure's architecture. This is especially desirable for biomaterial that is used for bone repair, since osteoclasts can then migrate into the material and use it as scaffolding for the construction of new bone. Porosity may be introduced into the material, for example, by the inclusion of porogen particles such as wax, alginate, etc. which may be removed through application of heat or which may dissolve after insertion in the body, and/or by creating stable bubbles which act as porogens, yielding micro- and macroporous structures with controlled architecture. When implanted into a recipient, such structures allow cells to infiltrate, and to differentiate into specific tissue within the scaffolding provided by the structure. In other embodiments, nanopores are introduced in order to allow the material to be impregnated with e.g. various drugs or other beneficial substances.

In addition, other beneficial materials may be incorporated into the biomaterials of the invention. Examples include but are not limited to: ions such as phosphate, calcium, etc. The deposition of ions may be due to the exogenous addition of these elements. Alternatively, they may be generated from media components by the action of electrodes (electrolysis) used to generate an electric field for controlling cellular motion. The presence of e.g. phosphate and calcium is especially advantageous when the biomaterial is intended for use as scaffolding to produce new bone growth, since these ions induce hydroxyapatite crystal growth, as well as to promote cell adhesion and the binding of growth factors. The mimetic biocompatible materials produced by electromagnetic biofabrication can be used for a variety of purposes, including but not limited to: as customizable implants, biocompatible coatings, biomedical devices or health care products, organ regeneration. The materials may be used as scaffolding for cell proliferation and differentiation, including stem cell proliferation and differentiation. For example, the mimetic biocompatible material may be inserted into cartilage, meniscus, tendons, ligaments or bone to support cell colonization in vivo. Infiltration of the biostructures by one or more cell types of interest may occur after the material has been implanted into a recipient (in which case the material acts as a scaffolding). Alternatively, porous forms of the material may be infiltrated by one or more cells of interest (e.g. autologous cells) prior to implantation, in which case the material is used as both a scaffolding and as a delivery device for seeding the cells. In addition, the material, if porous, may be impregnated with other beneficial substances prior to implantation.

The invention is further illustrated in the following Examples, which should not be construed so as to limit the invention in any way.

### EXAMPLES

### Example 1

Demonstration of control of motion of biopolymer extruding cells

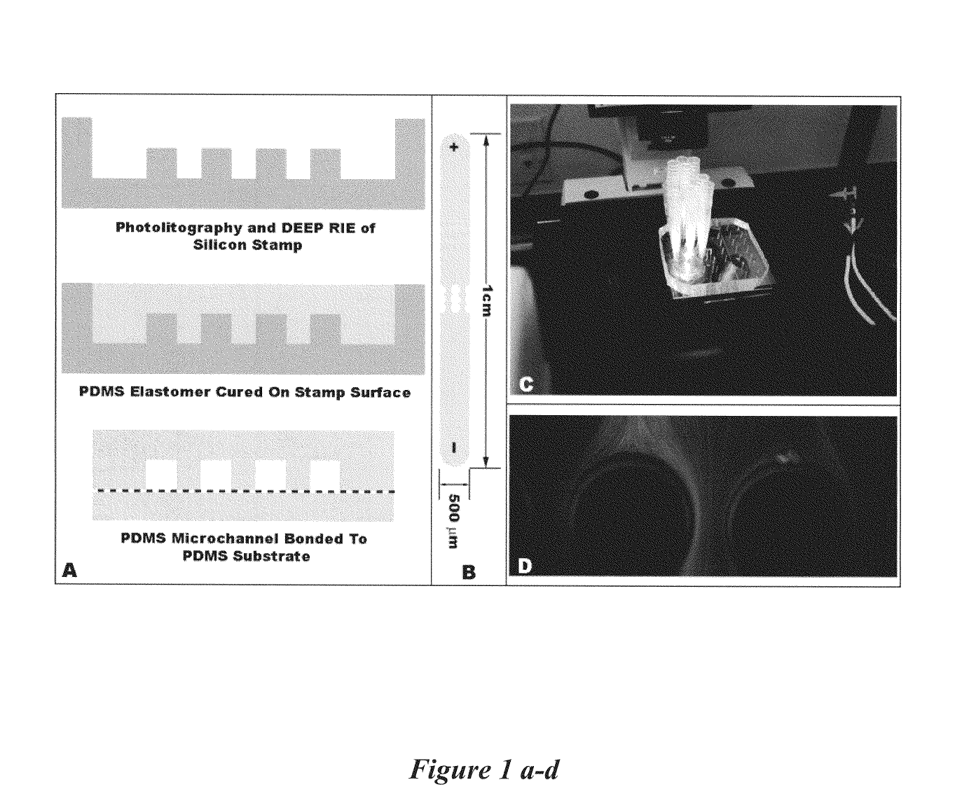

The key parameter towards tailor making properties of materials made by cells and bacteria is control of the biofabrication process which includes control of cell motion and proliferation. Experiments using Acetobacter xylinum were carried out in the devices which were produced using process shown in FIG. 1A. FIG. 1B shows dimensions of the device and FIG. 1 C shows device in use. The bacteria were controllably guided down the channel electrokinetically as is seen in FIG. 1D. The experiments showed that their behavior is similar to other bacteria studied in the devices of the invention. The motion of bacteria in the devices is governed by the electrophoretic, dielectrophoretic, and drag forces acting on them. The absolute velocity of the cell can be obtained by balancing these forces and solving for the velocity embedded in the force term due to Stoke's drag. The electrophoretic force impacted on the particle under an applied electric field is due to the charge of the particle. Whereas the particle's dielectrophoretic induced velocity is given by the product of the gradient of the electric field squared with the dielectrophoretic mobility. The motion conditions are configurable by adjusting the electric field distribution through modifying the channel geometry and the applied field. To control the motion of cells, such as bacteria A. xylinum to applied electric fields, we created microfluidic devices in polydimethylsiloxane (PDMS) (FIG. 1C). A silicon master stamp fabricated using standard photolithography and deep reactive ion etching (FIG. 1A). The stamp was then coated in PDMS and allowed to cure. The microfluidic channels produced in the stamping process were then irreversibly sealed to a flat sheet of PDMS by exposure to air plasma for 3 minutes in a PDC-001 Plasma Cleaner (Harrick Plasma, Ithaca, N.Y.). A. xylinum cells in culture media were then injected into the microfluidic channels and pressure was allowed to equalize. Platinum electrodes were then used to apply small electric fields across the channels inducing electrokinetic and dielectrophoretic forces that guided the bacterial cells as they produced cellulose nanofibers.

The bacterial strain employed was Acetobacter xylinum subsp.sucrofermentas BPR2001, trade number 700178™, from the ATCC. Fructose media with an addition of corn steep liquid (CSL) was be used as culture media. For pre-cultivation, 6 cellulose-forming colonies were cultured for 2 days at 30° C. in a Rough flask (nominal volume, 300ml; working volume, 100 ml) yielding a cell concentration of 3.7×10⁶ cfu/ml. The bacteria were than liberated by vigorous shaking and inoculating in the desired amount into the culture media.

The movement of bacteria was controlled by manipulating the electrokinetic forces acting on them. When a bacterium was placed in a uniform electric field, the resulting velocity of the particle was calculated by:

\vec{Vₑₖ}=(μₑₒ+μₑₚ)\vec{E}

where \vec{E}is the electric field in which the particle exists, μₑₒ and μₑₚ are the electro-osmotic and electrophoretic mobilities of the fluid and particle respectively. As convention we defined

μₑₖ−μₑₒ+μₑₚ

where μₑₖ is the electrokinetic mobility of the bacteria in the growth medium. While μₑₖ is generally an intrinsic parameter of a given system, \vec{E} can be experimentally varied to effect movement.

Linear motion of cellulose producing bacteria was controlled by applying DC electric fields to the device inlet ports and inducing electrokinetic flow. The electrokinetic mobility was measured by recording the mean velocity of the bacteria within the straight channel as a function of applied field and solution conditions. FIG. 1D shows the progression of the bacteria labeled with BacLight™ (Invitrogen, Carlsbad, Calif.) through the channel.

### Example 2

Demonstration of controlled 2D morphology (alignment) of biopolymer deposition during linear motion of cells within an electric field.

To produce cellulose networks suitable for evaluation, larger fluidic environments were created using stamps made from cleaved pieces of silicon measuring 19×5×0.5 mm and placed on a glass substrate. Complex unidirectional and bidirectional motion of cellulose producing bacteria was controlled by applying DC and AC electric fields to specific device inlet ports and inducing electrokinetic flow and dielectrophoretic cell movement. When a cell was placed in a non-uniform electric field, the resulting velocity of the cell was calculated by:

\vec{Vₚ}=μₑₖ\vec{E}+μ_{DEP}\vec{V}(\vec{E}·\vec{E})

where \vec{E} is the local electric field and μ_{DEP} is the dielectrophoretic mobility. μ_{DEP} is a function of the cell size and electrical properties as well as the properties of the surrounding medium. While the effects of an alternating electric field results in no net movement of a cell due to electrokinetic forces, the dielectrophoretic forces acts on the cell regardless of time varying fields.

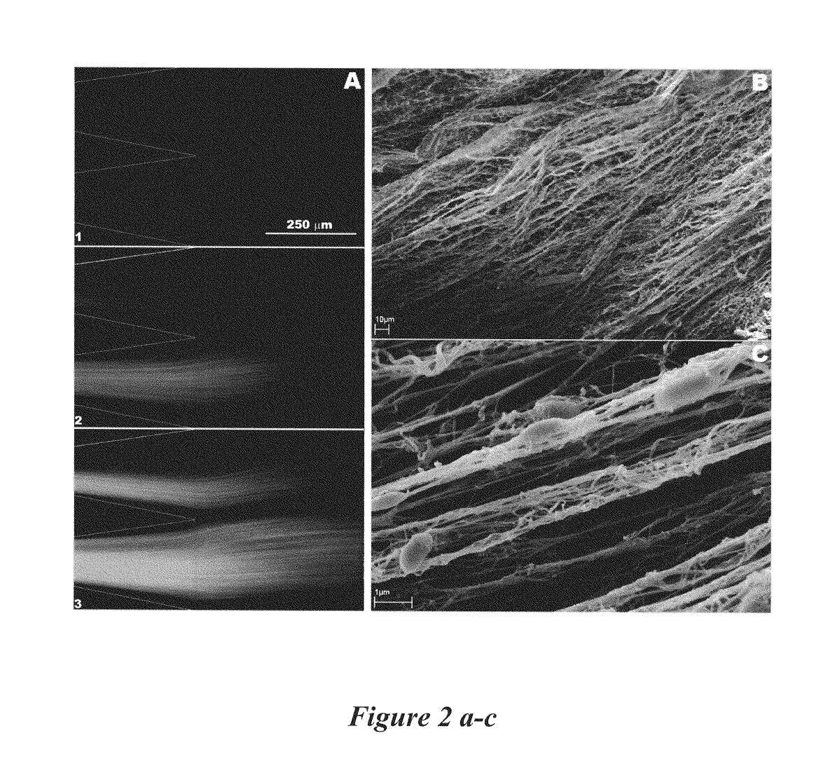

Without an applied electric field, the bacteria produce cellulose randomly resulting in the filling of the device with bacterial cellulose. Under high electrical fields, the bacteria are moved too quickly and cellulose production is switched off. However, there are experimental conditions in which the motion of the bacteria can be controlled while simultaneously producing cellulose. Specifically, when the bacteria were subjected to electric fields between 0.01V/cm and 1.0V/cm, while being guided through the chamber by electrokinetic and dielectrophoretic forces, the bacterial cells are being controlled with velocities on the order of 1 micron/s. FIG. 2A shows the progression of the bacteria labeled with BacLight™ (Invitrogen, Carlsbad, Calif.) through the device. Within this range, variations in the strength of the applied field change the morphology of the cellulose structure that is produced.

After 48 hours, cellulose production was halted by quenching the scaffolds in liquid nitrogen. The scaffolds were then freeze dried in a Labonco FreeZone 2.5 Plus (Labconco Corp., Kansas City, Mo.) freeze dryer for 48 hours without any further processing to leave the bacterial cells in situ. 5 nm of gold was then deposited on the scaffold and Field Emission Scanning Electron Microscopy (FESEM) was conducted at a working distance of 6 mm and 5 kV electron beam intensity using a LEO Zeiss 1550 FESEM (Carl Zeiss SMT, Oberkochen, Germany).

FIG. 2B shows an FESEM image of the cellulose produced under 0.303 V/cm in those which interwoven strands of nanocellulose fibrils are aligned in the direction of the applied electrical fields to which the bacteria were exposed. Increasing the field strength to 0.45V/cm produces a more finely stranded cellulose structure (FIG. 2C). The ellipsoid shaped particles on top of the strands in FIG. 2C are the bacteria which have been fixed to the cellulose fibers during the freezing process. Inspection of the branching nanofiber network shows that mitosis continues as the bacterium was guided through the microchannel creating an interweaved structure in which all of the nanofibers project in the same direction. The results in the FIG. 2 B and C clearly show that the orientation of cellulose fibers and the architecture of the network can be predictably controlled using electric fields.

### Example 3

Aligned fibrils have better mechanical properties

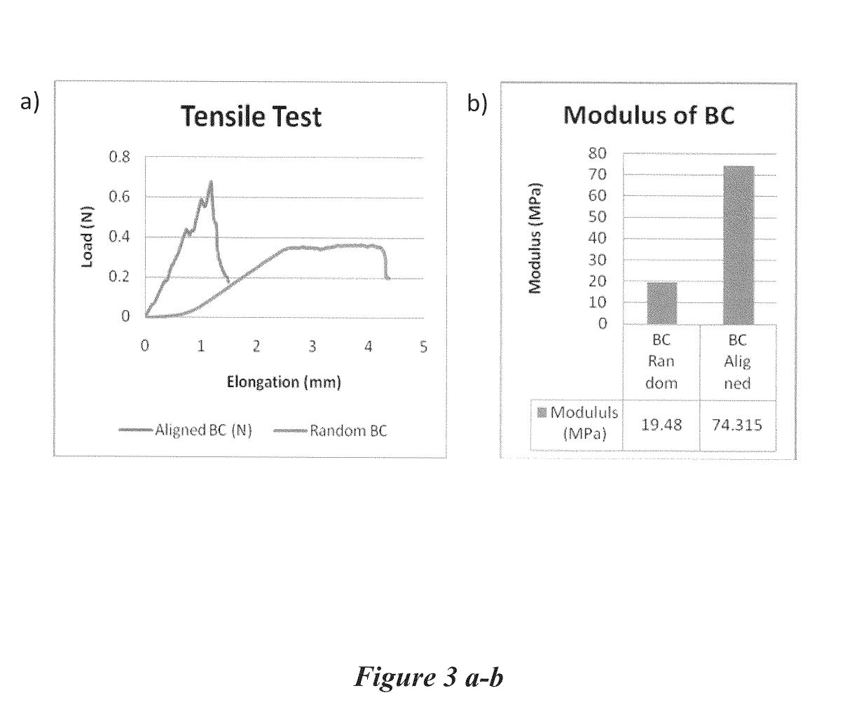

The mechanical properties of aligned BC fibrils (such as produced in Example 2, have been evaluated by tensile testing in the wet state and compared with a random network. Never dried samples were washed in 0.1 molar NaOH for 8 hours at 60° C. and stored in DI water until use. Instron tensile testing machine equipped with liquid chamber (Model Biopulse) was used to perform tensile test at 37° C. in simulated body fluid with an approximate strain rate of 10%. FIG. 3A shows tensile testing data (load versus elongation) on aligned BC fibrils compared with random BC network. It is clearly seen that aligned fibrils can take up more load compared to the same amount of randomly organized nanofibrils. The slope of the load displacement curve is much higher for aligned fibrils which is the evidence of higher stiffness. FIG. 3B shows that modulus of aligned BC is higher compared with random BC.

### Example 4

Various 2D controlled fibril alignments

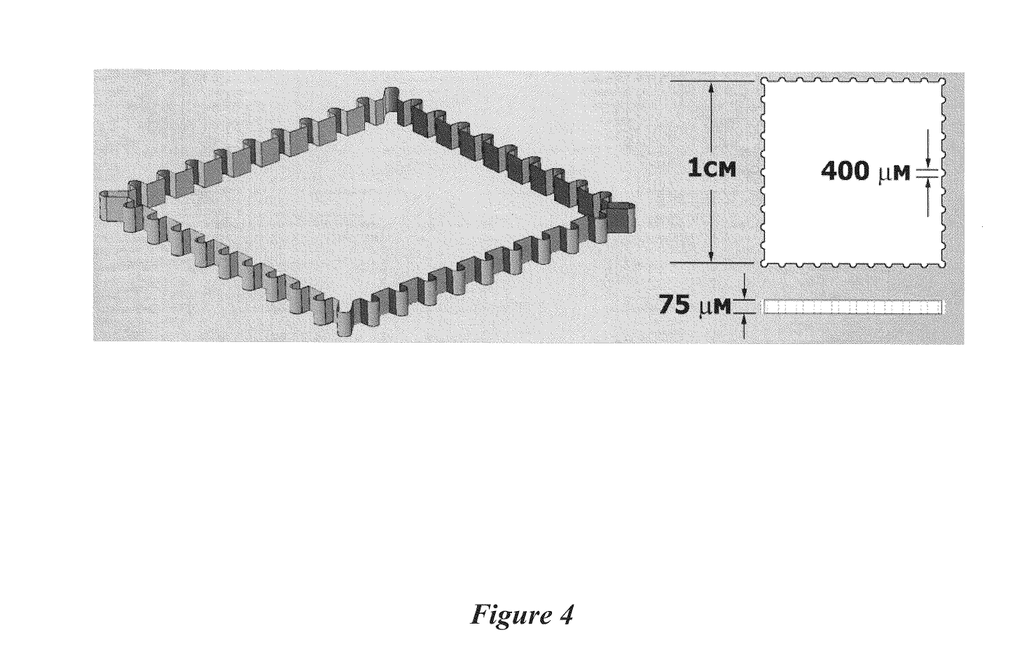

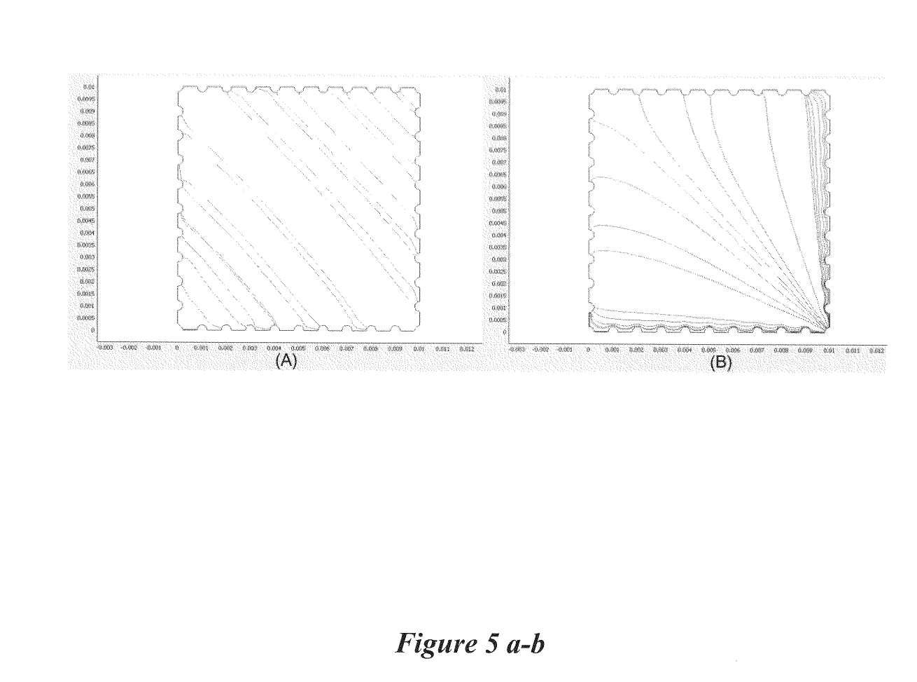

Various 2D patterns were produced as schematically shown in FIGS. 4-6. The device illustrated in FIG. 4 was used to create complex bidirectional patterns. This device has a 40 input electrode array around the perimeter and an open face to allow cellulose production at the liquid air boundary. Concentrated bacteria samples were injected into the desired input ports and the creation of complex cellulose patterns such as those shown in FIGS. 5A and B was demonstrated by selectively energizing perimeter electrodes.

Cellulose production can be controlled in an oscillatory motion to induce “crimping” (i.e. bending) in the BC cellulose scaffolds. Weaving or crosshatching of cellulose layers allows tuning of the structural properties of the scaffold.

The procedure was followed to move the bacteria left to right across the device. At specific times, the applied electric field was switched to move the bacteria right to left. After another predetermined length of time, the applied field was returned to its' original configuration. This process was repeated to produce an overall biomaterial structure with integrated crimped nanofibrils.

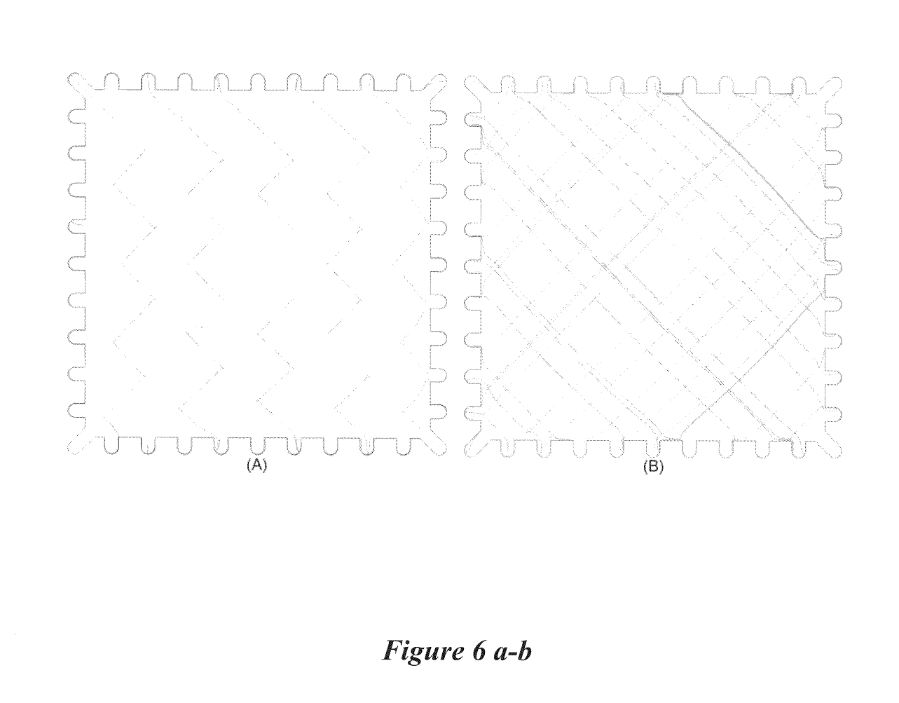

Additional experiments were conducted in which the applied fields were used to move the bacteria a desired length diagonally from left to right. The parameters were switched to move the bacteria diagonally from right to left and create a zigzag pattern as illustrated in FIGS. 6A and B. A more complex continuously time varying strategy was employed to create a sinusoidal pattern as the bacteria were moved across the device. To produce a cross hatched structure, the bacteria were moved left to right across the channel. Bacteria were then introduced at the top of the channel and moved to the bottom. The effect of an existing cellulose layer on movement and growth of a second perpendicular layer is also examined.

### Example 5

Demonstration of bacterial cellulose 3E scaffold fabrication to include pores and layers

Successive layers of BC scaffolding were grown by depositing a thin layer of growth media above a complete layer, thus forming a new solid-liquid-air boundary for scaffold production. Additionally the injection of temporary insulating particles (porogens), such as alginate or wax, allowed for the creation of complex porous 3D structures.

For substantial controlled tissue growth to occur, a multileveled supportive structure must be created. Metrics for creating 3D structures wre demonstrated by modifying the techniques developed in Example 1. Current laboratory experiments showed that BC layers formed most readily at the intersection of the solid, liquid, air boundary. The layers continue to develop across the remaining liquid-air boundary and remain attached to the outside solid boundary anchor points. It has also been observed that thin BC layers are neutrally buoyant, even when the layer is detached from its initial anchor points. When culture media is added above an existing BC layer, growth at that layer is impeded and cellulose production resumes at the new solid-liquid-air interface.

The device designed for Example 1 (shown in FIG. 1) was modified to accommodate the growth of multiple BC scaffold layers. Specifically, the channel depth was increased from 75 microns to approximately 1000 microns. A clean channel was initially primed with 0.025 mL of modified fructose culture media, to a height of approximately 250 microns. This provided sufficient fluid to support the scaffold layers and account for evaporation. Concentrated samples of Acetobacter xylinum were then injected into the channel and the procedures developed in Example 1 were followed to create a single layered BC scaffold at the solid-liquid-air interface.

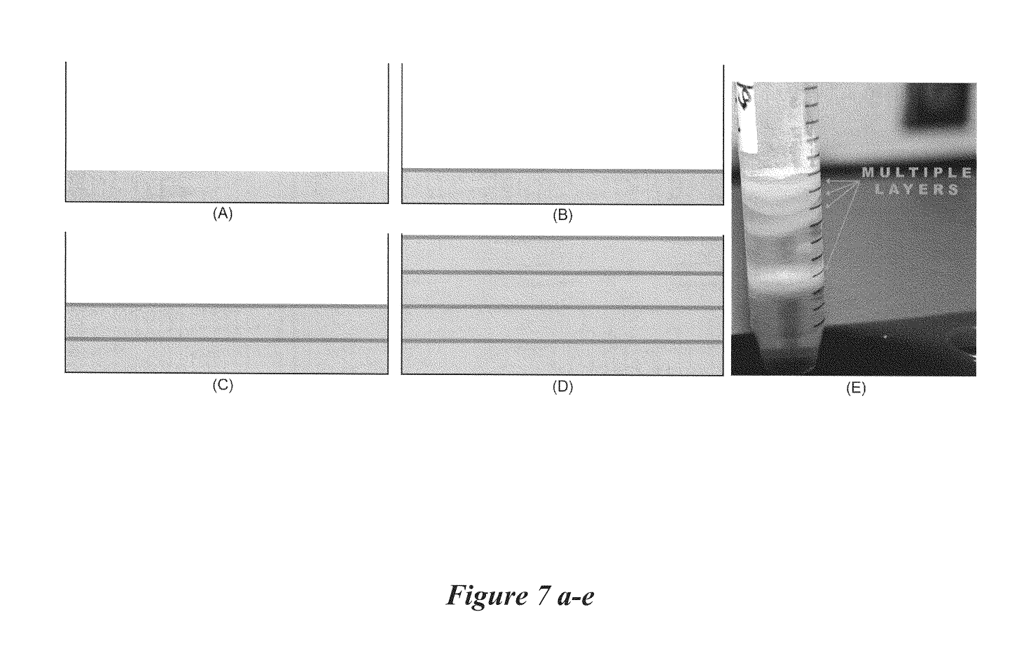

Upon completion of the first BC scaffold layer, an additional 0.025 mL of culture media was added to the top of the channel covering the existing layer. Concentrated bacteria samples were added to the channel and a new layer was grown at the liquid-air-interface. This process was repeated until the liquid-air interface approaches the top of the channel. The culture media was then completely drained from the channel leaving only the BC scaffold. Successful completion of 2, 3, and 4 tier scaffolds was followed by experiments using lesser quantities of growth media between successive layers with the goal of creating an experimentally infinite number of layers. This embodiment of the invention is illustrated schematically in FIGS. 7A-E, and actual experimental results are shown in FIG. 7E.

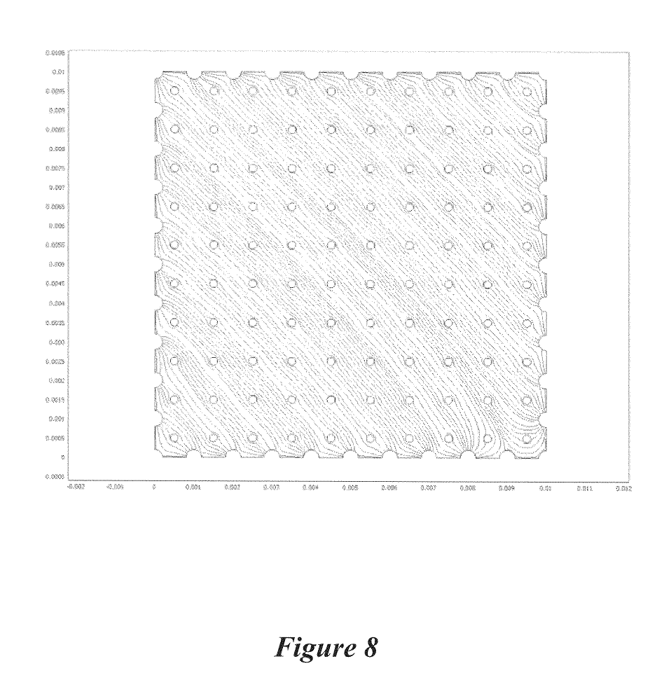

Injection of alginate and wax particles prior to growth of BC results in porous scaffold layers after the particles are dissolved in alkali or melted and removed. This embodiment is depicted schematically in FIG. 8. Physiological phenomena such as cell invasion, vascularization and nutrient transport as well as mechanical properties are all influenced by the overall geometry and porosity of the system. To mimic the complex porous structure found in the extracelular matrix (ECM) of many tissues, porogen particles are introduced into the channel to impede cellulose production in specific regions. Simulations and experimental results show that particle motion is not impeded by insulating structures at low voltages.

### Example 6

Oxygen formed by electrolysis stimulates scaffold production and can even create highly porous materials

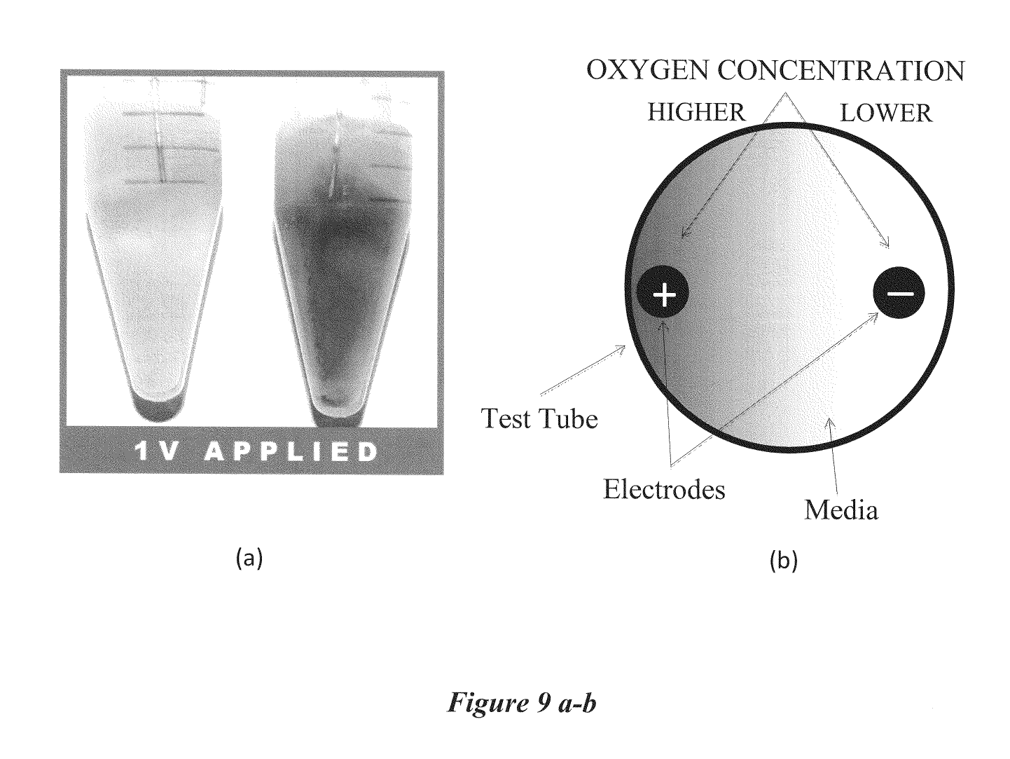

The experiment conducted in the tube with 1V applied showed that the production of cellulose is greatly enhanced due to oxygen generation through the electrolyses of the media (FIGS. 9 A and B). The blue dye is added to the tube on the right side to further visualize the process of 3D structure growth due to the increased oxygen concentration (FIG. 9A, and illustrated schematically in FIG. 9B).

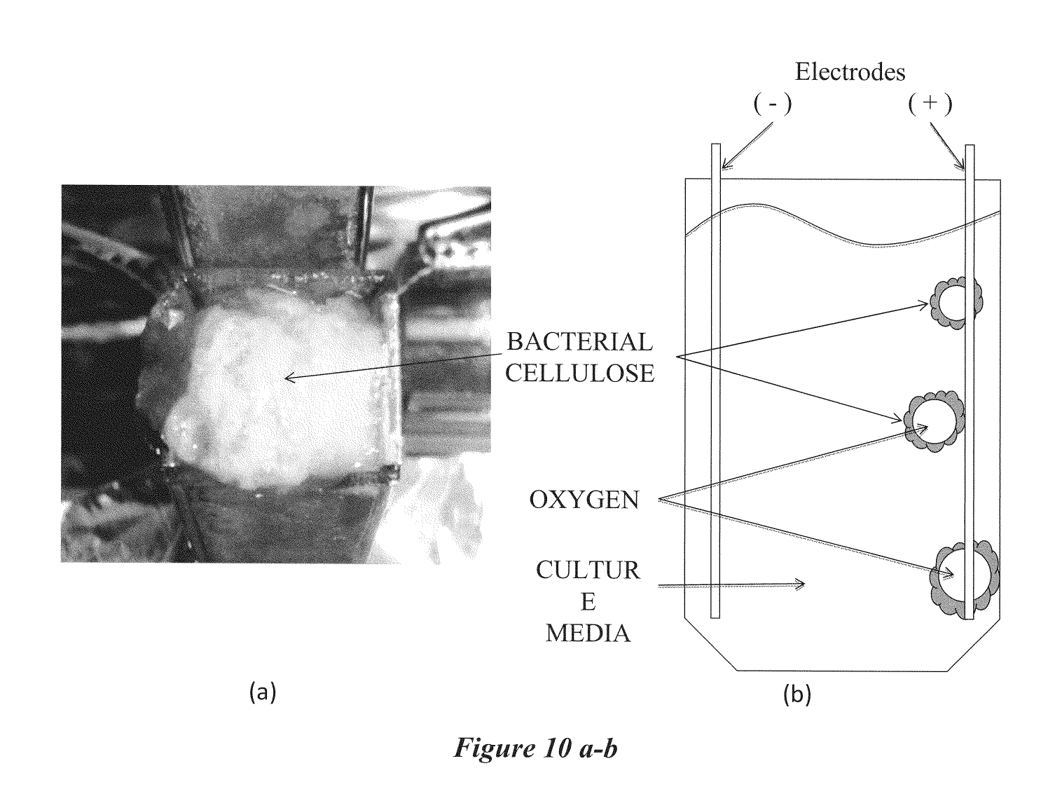

In another experiment the use of oxygen production to both enhance biofabrication and also as a porogen to produce highly micro and macroporous structures was evaluated. Conductive aluminum tape was used to create electrodes down two edges of lcm square plastic cuvettes (schematic illustration in FIG. 10B). The cuvettes were then filled ¾ with BC culture media inoculated with A. xylinum. Voltages ranging from 5-20 V DC were then applied to induce electrolysis and increase the oxygen content of the media. The bacteria produced cellulose around the oxygen bubbles. Surfactants such as plant based oils (olive oil) and variations in electric field strength can be used to modify the bubble properties such as diameter and persistence time, and hence to further modify the pattern of collagen production. Actual collagen generated is depicted in FIG. 10A.

### Example 7

Ions deposited during electrical discharge improve cell adhesion and cell differentiation

Phosphate: BC Culture media was modified by adding 25% PBS (Phosphate buffer solution). Micro chambers measuring 4.5 cm×0.5 cm×500 microns were filled with the modified media and subjected to 4V for 1-4 days (48 hours actual). Samples were then rinsed in NaOH for 8 hours at 60° C. then stored in DI water until use. Phosphate ions were detected on the surface of nanofibrils using EDS (Energy Dispersive X-ray Spectroscopy). Such modified fibrils were able to induce crystallization process of calcium deficient hydroxyapatite when samples were exposed to simulated body fluid. Osteoprogenitor cells colonized and attached strongly to such modified surfaces and differentiated into osteoblasts as shown by production of osteoblast specific proteins.

### Example 8

Preparation of customizable meniscus implant with microweaver using computer controlled biofabrication.

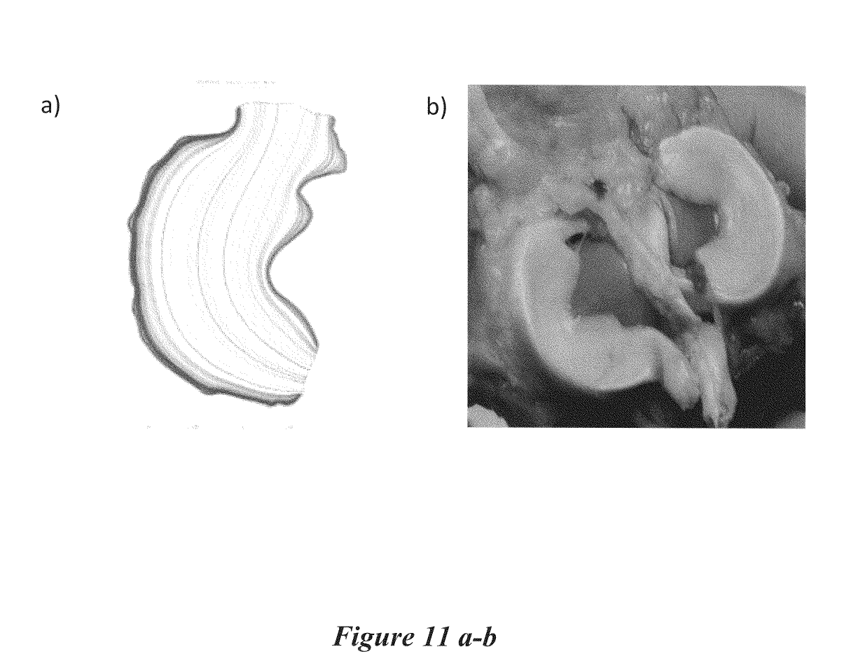

Bacteria tend to produce layers in a 2D-mode. The layers can be separated and this is a key to the control of 3D-dimensional architecture. The microweaver looks like a printing device and layer by layer can be weaved using a dielelectrophoretic field. This technology can be used to demonstrate computer aided fabrication of a three-dimensional network with good mechanical properties. The dielectrophoretic microweaver was created by stamp curing elastomer. Device stamps were micromachined into cast aluminum in the shape of the meniscus and silicon elastomer chambers was produced as previously described. Electric fields were applied at specific points within the chamber as determined by numerical simulations to produce cellulose scaffolds with the fiber alignments determined as shown in FIG. 11A. Aligned cellulose scaffolds as thick as 500 microns have been successfully created. Experimental conditions were varied to achieve maximum layer thicknesses and successive layers are stacked to create a total cellulose meniscus implant.

### Example 9

Cells migrate in porous structures and regenerate tissue

MC3T3-E1 osteoprogenitor cells bellow passage 20 were seeded onto the scaffolds in growth medium containing eMEM (Eagle's minimal essential medium, Invitrogen, Gaithersburg, MD, USA), 10% fetal bovine serum (FBS) (Gemini Bio-Products, Calabasas, Calif., USA) and I% antibiotic; antimycotic solution (Invitrogen). The following day, denoted as day 0, growth medium was replaced with differentiation medium (growth medium supplemented with 0.13 mM L-ascorbic acid 2-phosphate and 2 mM 13-glycerophosphate (Sigma)). Cells were grown in an incubator at 37° C. in 5% CO₂ and 95% relative humidity. The culture medium was changed every third day. Cell migrated into pores and after 10 days started to differentiate and produce extracellular matrix.

### Example 8

Stacking of multiple layers to create a complex 3D structure.

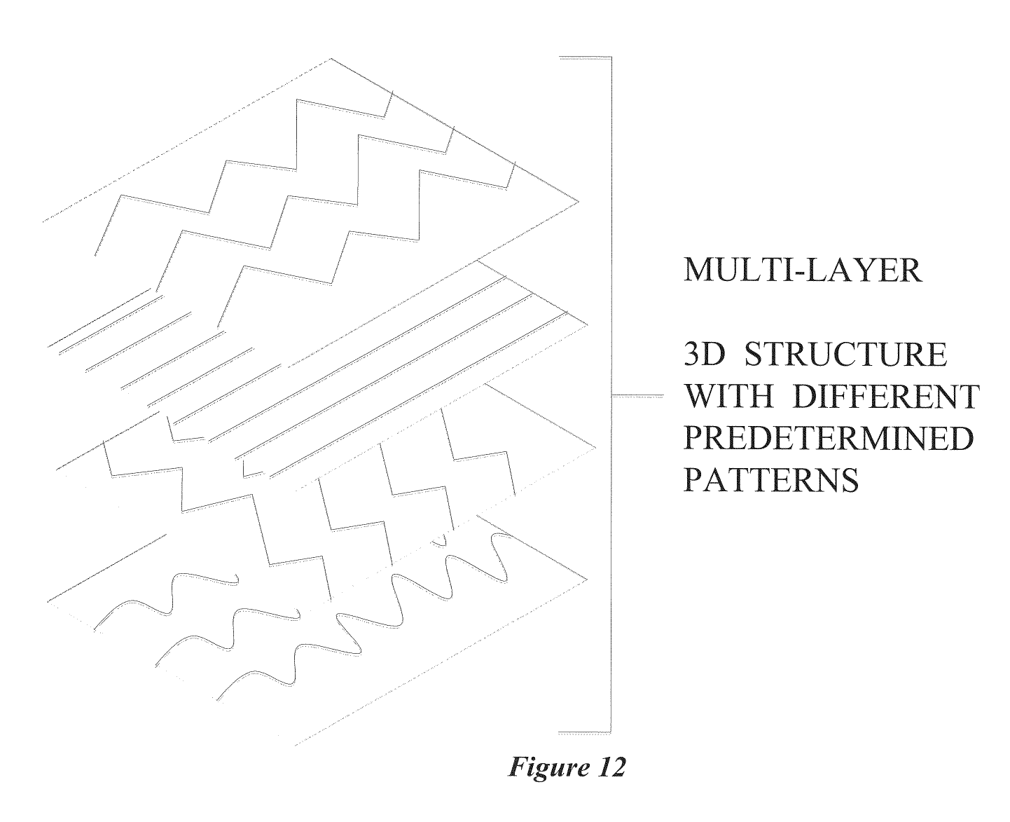

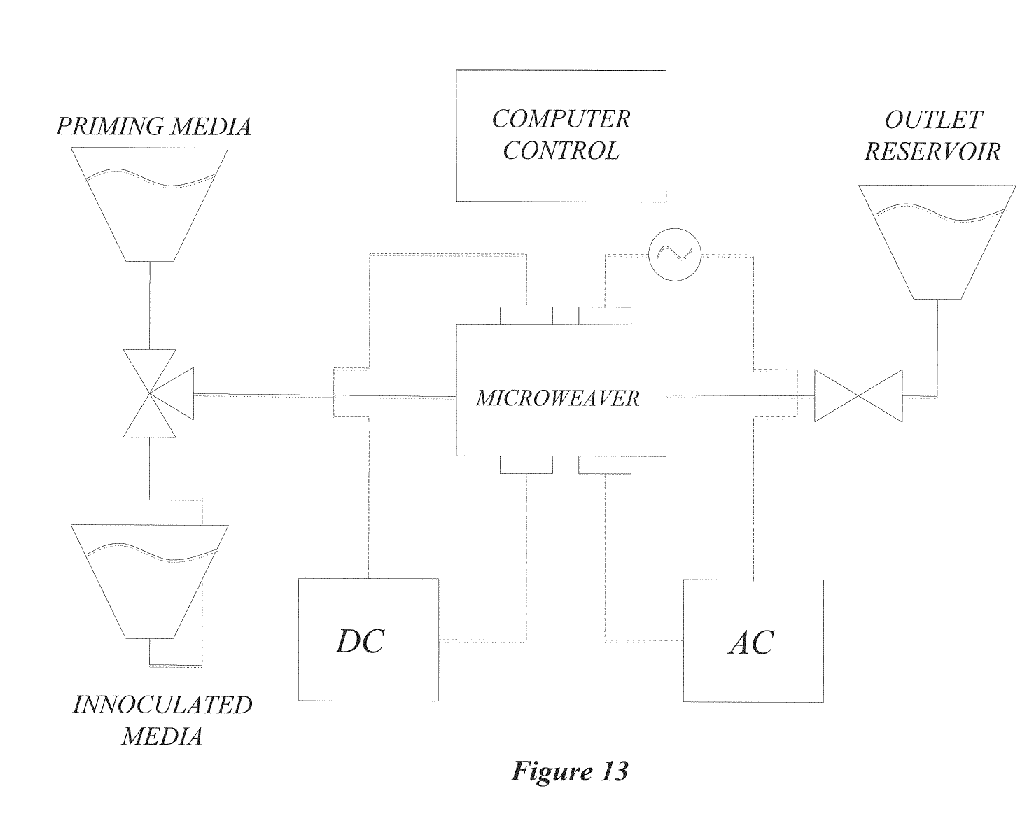

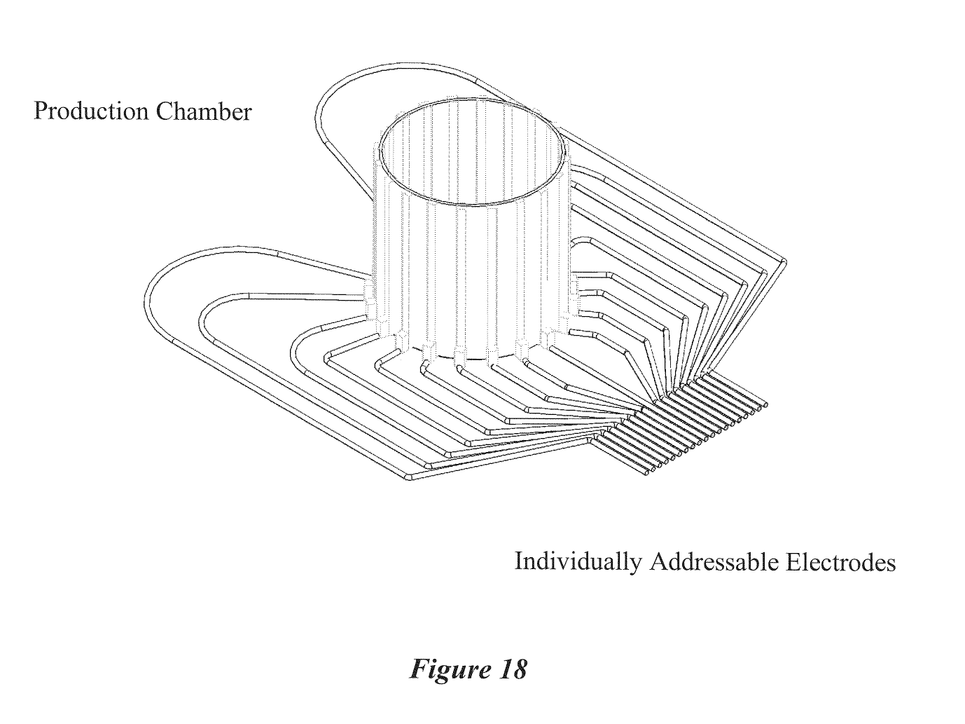

Multiple 2D layers are produced using a computer controlled microweaver setup as shown in FIG. 13. A production chamber with individually addressable electrodes, as will be shown in FIG. 18, is first filled with priming media from an inlet reservoir. Once the production chamber is primed, a valve (triangles in FIG. 13) is turned to allow inoculated media to enter the chamber. This media contains living polymer-producing cells. Computer controlled AC and DC power supplies then energize specific electrodes to induce electrokinetic, electrophoretic, and dielectrophoretic forces which guide the cells to create layers with prescribed fiber orientations. Individual layers are created in separate chambers. The layers are then stacked to form a multi-layer 3D structure with different predetermined patterns as shown in FIG. 12. Each sequential layer may have the same or different fiber alignment as the previous layer. The mechanical properties of this structure may be tuned to be used for a variety of applications. The second layer from the top, for example, will have high tensile strength in the direction the fibers are aligned in and low tensile strength in the alternate direction. Stacking this layer with the other three will provide additional tensile strength in the weak direction and provides some elasticity to the structure. Configurations such as this provide mechanically relevant structures for organs such as the knee meniscus, FIG. 11b, which has three regions of with distinctly different fiber alignments.

### Example 9

A chamber with insulating pillars and barriers for creating cohesive three dimensional scaffolds

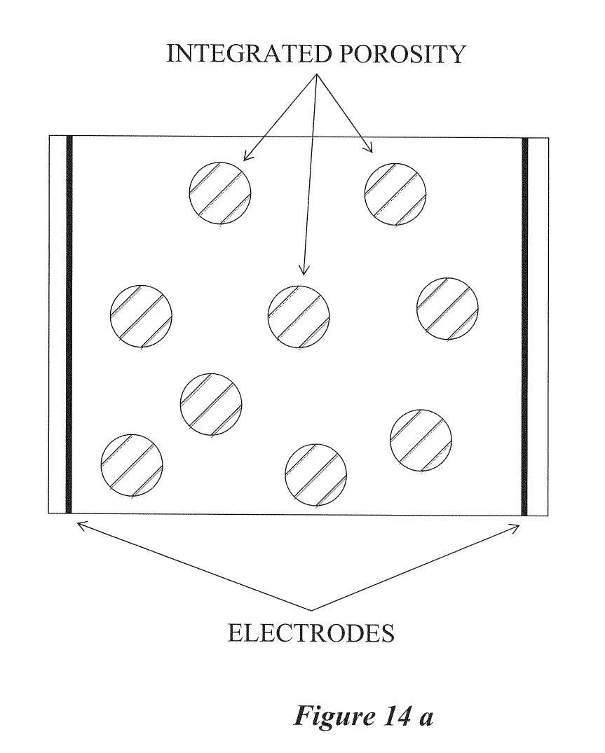

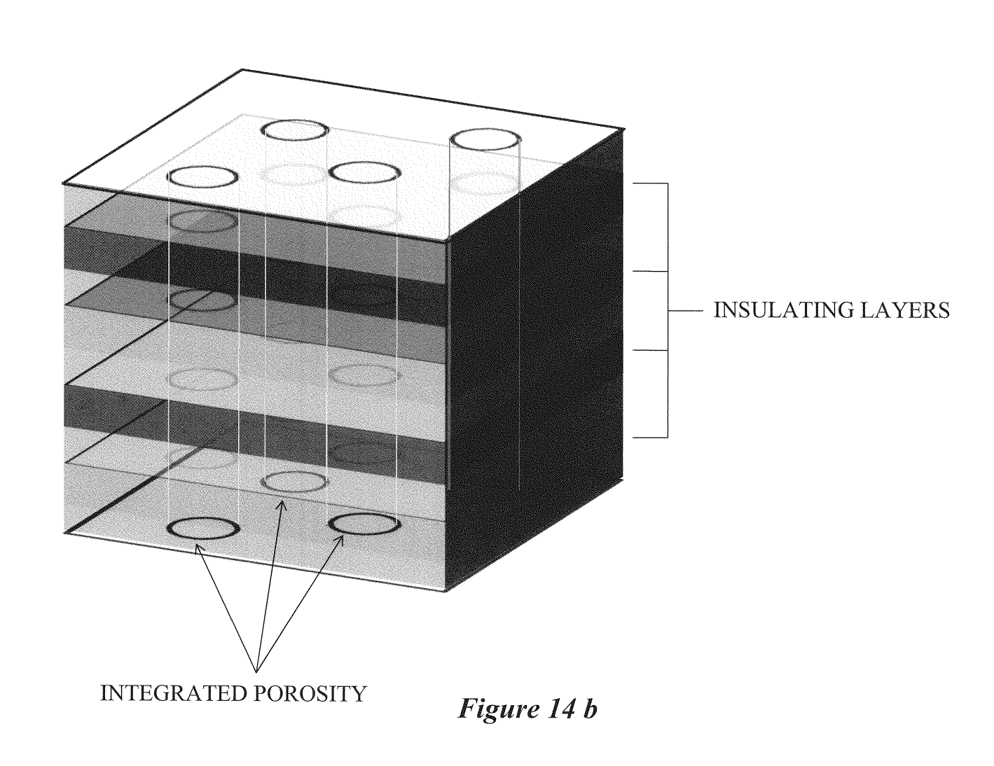

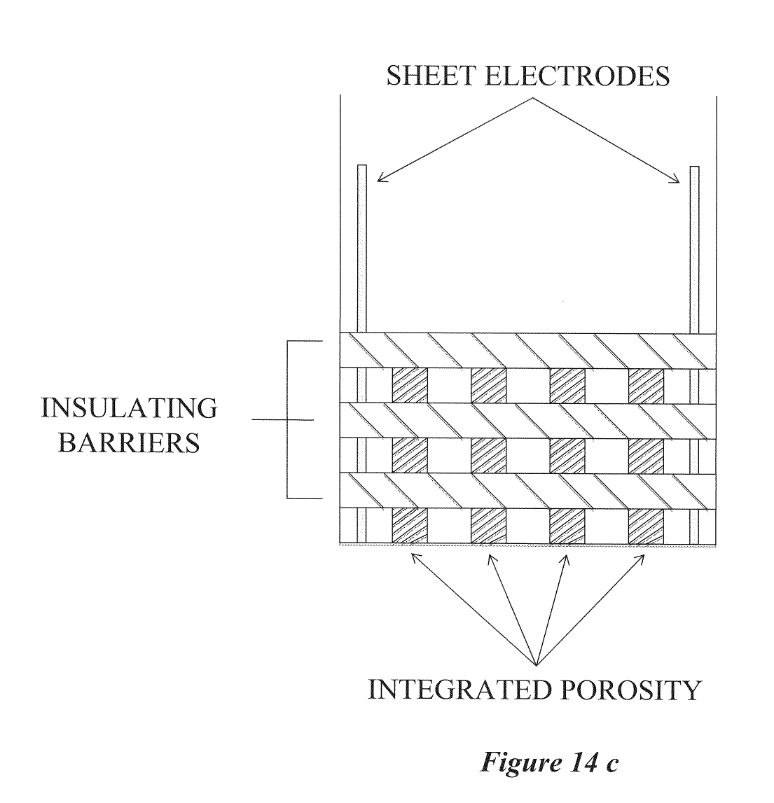

A material such as alginate is used to create insulating barriers and integrated porosity within the culture media as shown in FIGS. 14a, 14b, and 14c within a chamber created as described in Example 1. The insulating barriers serve three purposes, first to impede the growth of cellulose in prescribed locations, second to form an electrically insulating barrier between successive layers and lastly, to induce non-uniformities in the applied electric field and induce DEP forces. These structures are created via 3D printing or through pipetting and when removed in post processing, provide an integrated porosity and conduits for vasculature in the cellulose structure created. The electric field applied to each layer using sheet electrodes and may be different to create a scaffold with multiple layers with different fiber alignments.

### Example 10

A chamber in which dep forces control bacteria to creates sinusoidal fiber patterns

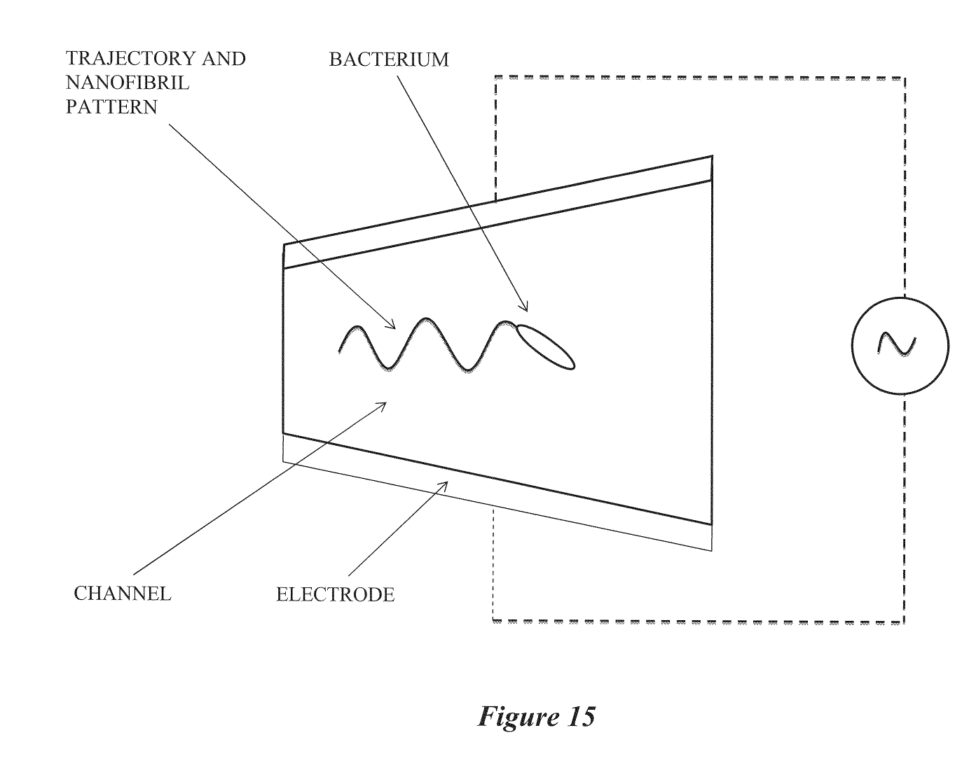

An asymmetric chamber created as described in Example 1 with electrodes located on two opposing sides as shown in FIG. 15 is used. When a low-frequency AC electric field is applied across the electrodes a field gradient is produced within the chamber. This gradient induces dielectropohretic forces on the bacteria along the length of the device. Additionally there is a potential difference across the channel between the electrodes inducing an electrokinietic force on the cells and they move up and down in the 2D plane due to the AC field. The net effect is control over the bacterium trajectory that produces a cellulose nanofibril pattern in the shape of a sine wave. The frequency of the applied field can be adjusted to change the amplitude of the fiber pattern deposited. The resultant scaffold layers can be integrated into a 3D structure as described in Example 8.

### Example 11

Halting cellulose production by inducing irreversible electroporation using insulating barriers.

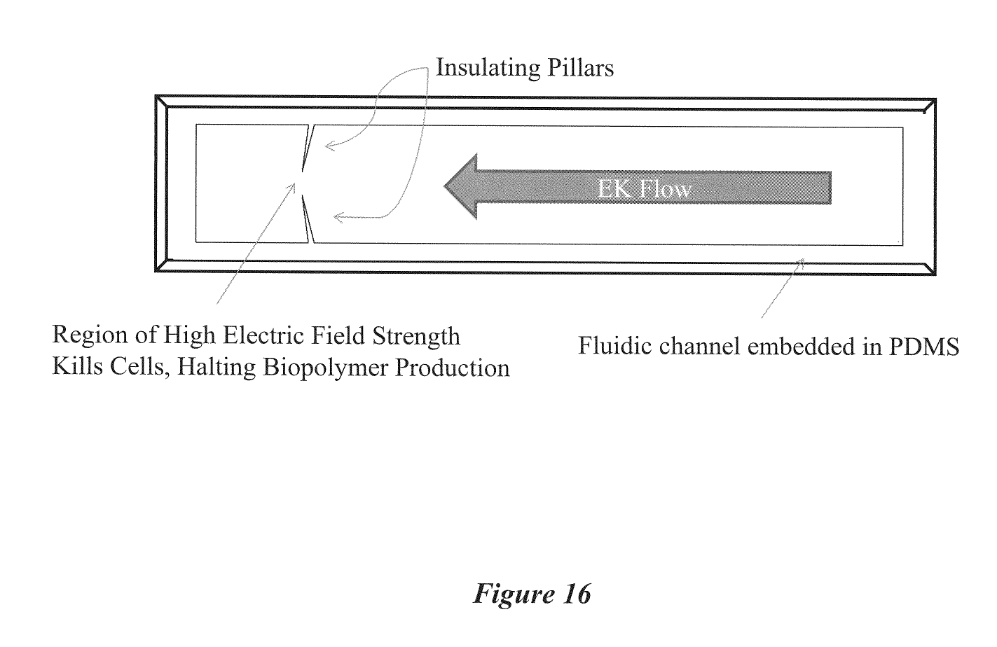

A fluidic chamber embedded in PDMS is created as described in Example 1 having insulating pillars, which creates a region of high electric field strength that kills cells, halting biopolymer production as shown in FIG. 16. The electric field within this region is large enough to induce irreversible electroporation and kills the cell, therefore halting cellulose production. The electric field external to this region is sufficient to guide the bacteria using EK flow, but not large enough to harm the cells.

### Example 12

Halting cellulose production by inducing irreversible electroporation by inducing a voltage spike.

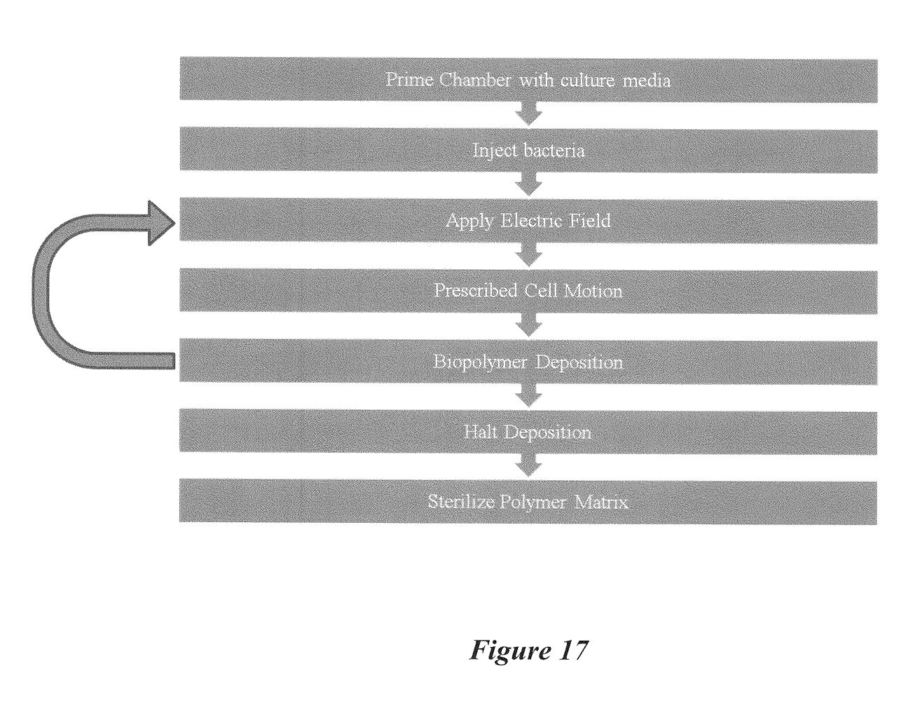

A fluidic chamber is created as described in Example 1 and polymer deposition and control are achieved as described in previous examples. An electric field large enough to cause a voltage drop of 1V or more across each cell in the camber is then induced within the chamber through a single spike or through a series of pulsed waves. All cells within the chamber are irreversibly electroporated and die, halting cellulose production. The entire process is shown in the Flow chart depicted in FIG. 17.

### Example 13

A method to create scaffolds with multiple fiber orientations within one chamber.

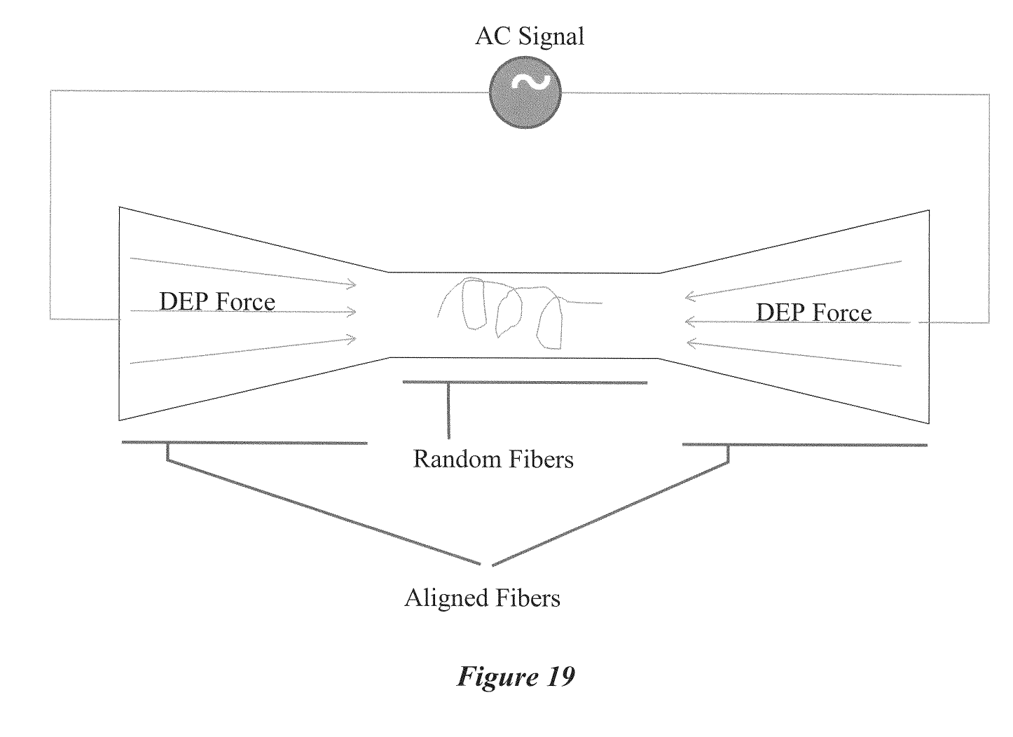

A fluidic chamber is created as described in Example 1 which has two trapezoidal regions separated by a rectangular region as shown in FIG. 19. An AC signal is applied across the ends of the channel inducing a DEP force in the trapezoidal regions and no net force in the rectangular region. The net motion of the cells in the trapezoidal regions is aligned and linear towards the center of the camber. The cells in the rectangular region produce a random network. The net result is a scaffold with three distinct regions from left to right, an aligned fibers region, then a random fibers region, then another aligned fibers region.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of producing a predetermined pattern of ordered biopolymers comprising the steps of
providing biopolymer-extruding cells in a liquid medium under conditions suitable for extrusion of biopolymers into said liquid medium by said biopolymer-extruding cells; and
applying an electromagnetic field to said liquid medium in a manner that causes said biopolymer-extruding cells to move according to said predetermined pattern while extruding said biopolymers, thereby forming said predetermined pattern of ordered biopolymers.

2. The method of claim 1, further comprising the step of varying said electromagnetic field.

3. The method of claim 1, wherein said predetermined pattern is three-dimensional.

4. The method of claim 1, further comprising the step of generating said electromagnetic field by suspending electrodes in said liquid medium.

5. The method of claim 4, wherein said electrodes are operated in a manner which produces oxygen.

6. The method of claim 4, wherein said electrodes are operated in a manner which produces ions from media components.

7. The method of claim 1, wherein movement of said biopolymer-extruding cells in said applied electromagnetic field is unidirectional.

8. The method of claim 1, wherein movement of said biopolymer-extruding cells in said applied electromagnetic field is bidirectional.

9. The method of claim 1, further comprising the step of halting extrusion of said bioplymers by said bacteria.

10. The method of claim 9, wherein extrusion of said biopolymers is halted by subjecting the biopolymer-extruding cells to an applied electric field sufficient to induce death.

11. The method of claim 10, wherein said applied electric field is sufficient to induce a 1V or greater drop in potential across a cell membrane, thereby inducing irreversible electroporation.

12. A method of claim 10, wherein said applied electric field is sufficient to lyse said biopolymer-extruding.

13. The method of claim 1, wherein movement of said biopolymer-extruding cells in said applied electromagnetic field traces a curve.

14. The method of claim 1, wherein said predetermined pattern of ordered biopolymers forms at a gas-liquid interface of said liquid medium.

15. The method of claim 1, wherein said biopolymer-extruding cells are bacterial cells.

16. The method of claim 15, wherein said bacterial cells are of a as species selected from Acetobacter, Agrobacterium, Rhizobium, Pseudomonas and Alcaligenes.

17. The method of claim 16, wherein said cells are Acetobacter xylinum or Acetobacter pasteurianus.

18. The method of claim 1, wherein said biopolymers are bacterial cellulose.

19. The method of claim 1, wherein said electromagnetic field is an electric field.

20. The method of claim 19, wherein said electric field is from 0.1V/cm to 100V/cm.

21. The method of claim 2, wherein said step of varying said electromagnetic field is carried out by a programmed computer.

22. The method of claim 1, wherein said predetermined pattern includes pores.

23. The method of claim 22, wherein said pores are of a size sufficient to allow infiltration of animal or human cells into said pores.

24. A device for producing a predetermined pattern of ordered biopolymers said device comprising
a container for containing biopolymer-extruding cells in a liquid medium under conditions suitable for extrusion of biopolymers into said liquid medium by said biopolymer-extruding cells; and
means for applying an electromagnetic field to said liquid medium in a manner that causes said biopolymer-extruding cells to move according to said predetermined pattern while extruding said biopolymers, thereby forming said predetermined pattern of ordered biopolymers.

25. A method of forming a predetermined pattern of ordered biopolymers comprising the steps of
providing biopolymer-extruding cells in a liquid medium under conditions suitable for extrusion of biopolymers in liquid at or near a liquid-oxygen interface, by said biopolymer-extruding cells;
suspending electrodes in said liquid medium; and
operating said electrodes in a manner which generates one or more liquid-oxygen interfaces in said liquid media, whereupon said biopolymer-extruding cells extrude said bioplymers in said liquid at or near said one or more oxygen-liquid interfaces in said predetermined pattern of ordered biopolymers.

26. A device for producing a predetermined pattern of ordered biopolymers in vitro, said device comprising
a container for containing biopolymer-extruding cells in a liquid medium under conditions suitable for extrusion of biopolymers in liquid at or near a liquid-oxygen interface, by said biopolymer-extruding cells; and
means for generating one or more liquid-oxygen interfaces in said liquid media in a manner that causes said biopolymer-extruding cells to extrude said bioplymers in said liquid at or near said one or more oxygen-liquid interfaces in said predetermined pattern of ordered biopolymers.

27. A medical implant, comprising a polymeric material at least a portion of which includes a predetermined pattern of ordered biopolymers including one or more fibrils oriented in a manner which provides a specified tensile strength in at least one dimension.

28. The medical implant of claim 27, further comprising at least one opening which passes through said polymeric material.

29. The medical implant of claim 27, wherein said polymeric material is configured in a form of a human meniscus or other cartilage tissues.

30. The medical implant of claim 27, wherein said polymeric material is configured in a form suitable for a bone graft.

31. The medical implant of claim 27, wherein said polymeric material is configured in a form of tendons or ligaments.

32. The medical implant of claim 27, wherein said polymeric material is configured in a form for neural network support.

33. A polymeric material at least a portion of which includes a predetermined pattern of ordered biopolymers including one or more fibrils oriented in a manner which provides a specified tensile strength in at least one dimension.

34. The polymeric material of claim 33 wherein said predetermined pattern is in the form of a weave.

35. A multilayered polymeric material including a plurality of layers each of which includes at least one predetermined pattern of ordered biopolymers including one or more fibrils oriented in a manner which provides a specified tensile strength in at least one dimension.

36. Scaffold for tissue engineering, cell differentiation and organ regeneration, comprising a polymeric material at least a portion of which includes a predetermined pattern of ordered biopolymers including one or more fibrils oriented in a manner which provides a specified tensile strength in at least one dimension and comprising at least one opening which passes through said polymeric material.
